(19) United States
(12) Patent Application Publication
(10) Pub. No.: US 2008/0242594 A1
(43) Pub. Date: Oct. 2, 2008

(54) Methods for Promoting Stem Cell Proliferation and Survival

(76) Inventors: Ronald D.G. McKay, Bethesda, MD (US); Andreas Androutsellis-Theotokis, Bethesda, MD (US)

Correspondence Address: KLARQUIST SPARKMAN, LLP, 121 S.W. SALMON STREET, SUITE #1600, PORTLAND, OR, 97204-2988, US

(21) Appl. No.: 12/066,075

(22) PCT Filed: Sep. 7, 2006
(86) PCT No.: PCT/US06/34988

§ 371 Date: Mar. 6, 2008

Related U.S. Application Data
(60) Provisional application No. 60/715,935, filed on Sep. 8, 2005.

(51) Int. Cl. A61K 38/28; C12N 5/02; C12N 5/08; G01N 33/574; G01N 33/53; A61K 38/18

(52) U.S. Cl. 514/3; 435/375; 435/366; 514/12; 435/7.1; 435/7.23

(57) ABSTRACT

It is disclosed herein that STAT3 phosphorylated at serine 727 is a key regulator of proliferation and survival of stem cells and precursor cells. Methods for increasing the survival and proliferation of stem cells and/or precursor are disclosed herein. In one embodiment, the method includes contacting a mammalian stem cell mammalian precursor cell with a JAK inhibitor, a p38 inhibitor, or both. Methods are also disclosed for increasing the survival and proliferation of neuronal precursor cells in a subject. The method includes administering a therapeutically effective amount of a Notch ligand and a growth factor. Methods are also disclosed for identifying an agent that increases the proliferation of stem cells and/or precursor cells. The method includes contacting a stem cell or a precursor cell with an agent of interest, wherein the stem cell or the precursor cell expresses STAT3; and determining the phosphorylation status of serine 727 of STAT3 in the cell. Phosphorylation of serine 727 indicates that the agent increases the survival and/or proliferation of stem cells and/or precursor cells. An isolated population of cells is disclosed, wherein the cells express nestin and STAT3, wherein serine 727 of STAT3 is phosphorylated.

### CROSS REFERENCE TO RELATED APPLICATION

This claims the benefit of U.S. Provisional Application No. 60/715,935, filed Sep. 8, 2006, which is incorporated by reference herein in its entirety.

### FIELD

This application relates to the field of stem cells, specifically to methods and agents that are of use to promote stem cell proliferation and survival.

### BACKGROUND

Stem cells have been identified in several somatic tissues including the nervous system, bone marrow, epidermis, skeletal muscle, and liver. Populations of stem cells are believed to be responsible for maintaining homeostasis within individual tissues in adult animals. The number of stem cells and their differentiation and proliferation must be tightly controlled during embryonic development and in the adult animal. Different somatic stem cells share the properties of self-renewal and multi-developmental potential, suggesting the presence of common cellular machinery in populations of stem cells.

Embryonic stem (ES) cells can proliferate indefinitely in an undifferentiated state. Furthermore, ES cells are totipotent cells, meaning that they can generate all of the cells present in the body (bone, muscle, brain cells, etc.). ES cells have been isolated from the inner cell mass of the developing murine blastocyst (Evans et al., Nature 292:154-156, 1981; Martin et al., Proc. Natl. Acad. Sci. U.S.A. 78:7634-7636, 1981; Robertson et al., Nature 323:445-448, 1986; Doetschman et al., Nature 330:576-578, 1987; and Thomas et al., Cell 51:503-512, 1987; U.S. Pat. No. 5,670,372). Additionally, human cells with ES properties have recently been isolated from the inner blastocyst cell mass (Thomson et al., Science 282:1145-1147, 1998) and developing germ cells (Shamblott et al., Proc. Natl. Acad. Sci. U.S.A. 95:13726-13731, 1998) (see also U.S. Pat. No. 6,090,622, WO 00/70021 and WO 00/27995).

Dopaminergic neurons have been generated from murine CNS precursor cells (PCT Application No. PCT/US99/16825; and Studer et al., Nature Neurosci. 1:290-295, 1998). These precursor-derived neurons are functional in vitro and in vivo and restore behavioral deficits in a rat model of Parkinson's disease. Even though the primary mesencephalic CNS stem cell culture can provide differentiated dopaminergic neurons suitable for use in cell therapy, the cell number provided by this method is limited, and cell survival is limited. Thus, a need clearly remains for alternate sources of these cells.

There is growing interest in the analysis of patterns of gene expression in cells, such as cancer cells and stem cells, using microarray technology. However, few studies have elucidated the complex network of signals that functions in stem cells to promote proliferation. The molecular pathways that result in the proliferation of stem cells are disclosed herein, as well as methods of increasing the proliferation of stem cells. These stem cells can be used for the treatment of a variety of disorders, including but not limited to neurological disorders, wound healing, hepatic disease, and diabetes, amongst others.

### SUMMARY

It is disclosed herein that contacting stem cells or precursor cells with either an inhibitor of Janus kinase (JAK), an inhibitor of p38, or both, increases the survival and/or proliferation of stem cells and precursor cells. In response to the JAK inhibitor and or the p38 inhibitor, the stem cells or precursor cells express STAT3 phosphorylated at serine 727. To increase proliferation and survival of stem cells and precursor cells, STAT3 can be phosphorylated at serine 727.

Thus, methods for increasing the survival and proliferation of stem cells and/or precursor cells are disclosed herein. In one embodiment, the method includes contacting a mammalian stem cell mammalian precursor cell with a JAK inhibitor, a p38 inhibitor, or a combination thereof. In another embodiment, methods are disclosed for increasing the survival and/or proliferation of stem cells and/or precursor cells that include the use of a Notch ligand or a Notch agonist.

Methods are also disclosed for increasing the survival and proliferation of neuronal precursor cells in a subject. The method includes administering a therapeutically effective amount of a Notch ligand and a growth factor. In one specific, non-limiting example, the method includes administering Delta and FGF-2. The method can include administration by intraventricular injection.

Methods are also disclosed for identifying an agent that increases the proliferation of stem cells and/or precursor cells. The method includes contacting a stem cell or a precursor cell with an agent of interest, wherein the stem cell or the precursor cell expresses STAT3; and determining the phosphorylation status of serine 727 of STAT3 in the cell. Phosphorylation of serine 727 indicates that the agent increases the survival and/or proliferation of stem cells and/or precursor cells.

An isolated population of cells is disclosed, wherein the cells express nestin and STAT3, wherein serine 727 of STAT3 is phosphorylated.

The foregoing and other features and advantages will become more apparent from the following detailed description of several embodiments, which proceeds with reference to the accompanying figures.

### BRIEF DESCRIPTION OF THE FIGURES

FIGS. 1a-1f are a set of images showing that Notch ligands stimulate survival in CNS stem cell (CNS SC) cultures. FIG. 1a is a bar graph throwing the effect of Notch activation by Jagged-1, Delta-4, or combination in the presence of FGF2 for 5 days on plating efficiency, colony size, and BrdU incorporation (following a 4 hour BrdU pulse), over control treatments (FGF2 alone) FIG. 1b is a graph showing lineage and death events in murine E13.5 cortical stem cell cultures in the presence of FGF2 or FGF2 and Delta-4 (“D4”), D114 inhibits cell death in real-time cell lineage experiments (“x” marks time of death). CNS stem cells from embryonic day 13.5 mouse cerebral cortex were plated following one passage and visualized in real-time for 36 hours (images were taken every 2 min). Lineage and death events are represented. Cells were cultured in a closed gas chamber at 37° C. and images of selected fields were taken every two minutes. FIG. 1c is a line graph showing accumulative real-time imaging representation of death even times over 36 hours in culture with or without Delta-4. FIG. 1d is a bar graph showing Delta-4 treatment did not affect cell cycle length. Cell cycle durations for the first two divisions are shown. FIG. 1e is a bar graph showing Delta-4 treatment of CNS SC cultures replaced the requirement for insulin in the medium in a plating efficiency assay. FIG. 1f is a set of digital images showing that Delta-4 treatment of CNS SC activated Akt by phosphorylation on Ser473 and Ser308 in the presence (top) and absence (bottom) of insulin in the culture medium, with a peak at 5 min post-treatment, as revealed by Western blotting analysis. In the graphs, data represent Means ±standard deviation (SD).

FIGS. 2a-2h are a set of images showing cilliary neurotrophic factor (CNTF) and Notch ligands stimulate phosphorylation of serine727 on Stat3. FIG. 2a is a digital image showing a dose response curve for a 30 minutes CNTF-induced STAT3 phosphorylation on Ser727 and Tyr705. FIG. 2b is a digital image showing treatment with high CNTF concentrations (20 ng/ml) in the presence of a JAK Inhibitor selectively blocked Tyr705 but not Ser727 phosphorylation. FIG. 2c is a bar graph showing low CNTF concentrations (0.01, 0.1 ng/ml), JAK inhibition, and high CNTF concentrations (20 ng/ml) in the presence of JAK inhibitor increased survival. Data shows cell survival under different pharmacological conditions, expressed as a percentage of FGF2 control, after 5 days. FIG. 2d is a digital image showing Notch activation induced phosphorylation of STAT3 on Ser727 in a time-dependent manner but did not induce Tyr705 phosphorylation. CNTF was used as a positive control for STAT3 phosphorylation. FIG. 2e is a digital image showing γ-secretase inhibition by DAPT blocked the Delta-4-induced phosphorylation of STAT3 on Ser727. FIG. 2f is a digital image showing STAT3 phosphorylation at Ser727 was increased by Jagged-1 and Delta-4 in a dose-dependent manner. FIG. 2g is a digital image showing JAK Inhibition reduced the levels of phosphorylation on p38 MAP kinase at Thr180/Tyr182. FIG. 2h is a bar graph showing p38 MAP kinase inhibition mimicked JAK inhibition in enhancing plating efficiency. In graphs, the data represent Means ±standard deviation (SD).

FIGS. 3a-3i are a set of images showing serine727 on Stat3 integrates second messenger pathways that control survival. FIGS. 3a-3b are digital images showing that STAT3-Ser727 phosphorylation following Notch activation was sensitive to inhibition of PI3 kinase by LY294002 and Src kinase by SU6656. FIG. 3c is a digital image showing that Notch activation caused the time-dependent phosphorylation of mTOR on Ser2448. FIGS. 3d-3e are digital images showing STAT3-Ser727 phosphorylation following Notch activation was sensitive to mTOR inhibition by rapamycin, Cam Kinase II inhibition by KN93 and KN62, but not the negative control product KN92. FIG. 3f is a bar graph showing the effect of kinase inhibitors on CNS SC colony formation. CNS SC can form all three CNS lineages, including astrocytes, oligodendrocytes and neurons. FIG. 3g is a digital image showing Notch activation by Jagged-1 treatment (200 ng/ml) induced the time-dependent activating phosphorylation of MSK-1 and LKB1 kinases, the inactivating phosphorylation of GSK3β, and the activating de-phosphorylation of β-catenin. FIG. 3h is a digital image of epifluorescence detection of cells transfected with different STAT3 plasmids at day 4. Scale bars, 100 μm. FIG. 3i is a bar graph showing CNS SC transfected with a mutant form of STAT3 that cannot be phosphorylated on Ser727 showed decreased survival 4 days post-transfection. Conversely, cells transfected with a mutant that cannot be phosphorylated on Tyr705 did not show a change in survival from wild-type. The data represent percentage of transfected cells alive at the end of 4 days, relative to cells transfected with wild-type STAT3 (Data represent Means ±SD).

FIGS. 4a-4i are a set of images showing nucleostemin, SHH and Hes3 are Stat3-Ser effectors. FIG. 4a is a digital image showing Notch activation by Delta-4 treatment of CNS SC cultures induced nucleostemin and SHH protein expression, with a peak between two and three days. FIG. 4b is a digital image showing treatments that induce Stat3-Ser727 only phosphorylation and survival (Delta-4, CNTF+JAK Inhibitor, low CNTF concentrations) also induced nucleostemin expression at 2 days post-treatment. FIG. 4c is a digital image showing RT-PCR analysis of Hes/Hey family member expression in E13.5 CNS SC cultures following Notch activation. FIG. 4d is a digital image showing Jagged-induced elevation of Hes3 mRNA was sensitive to the mTOR inhibitor rapamycin. FIG. 4e is a digital image showing CNTF blocked the Jagged-1-induced Hes3 mRNA induction. This blockade was reversible by simultaneous JAK Inhibition. FIG. 4f is a digital image showing transfection of CNS SC with Hes3a or Hes3b increased SHH but not nucleostemin protein expression, at 2 days. FIG. 4g is a digital image showing Hes3 mRNA and protein are concentrated in the adult mouse SVZ (mRNA preparations were made from adult mouse SVZ, whole brain, and whole brain after SVZ removal). FIG. 4h is a digital image showing immunocytochemical detection of adult rat SVZ stem cell cultures in the presence of FGF2 and Delta-4. Scale bars, 100 μm. Blue: DAPI. FIG. 4i is a digital image showing Delta-4 treatment increased plating efficiency and colony size of adult rat SVZ stem cells in the presence of FGF2.

FIGS. 5a-5g are a set of images showing Notch activation and JAK inhibition promote hES cell survival. FIG. 5a is a bar graph showing JAK inhibition and, to a lesser extent, Delta-4 treatment, increased plating efficiency of HSF6 hES cells plated as single dissociated cells within 6 days. FIG. 5b is a digital image showing mouse embryonic fibroblast (MEF)-conditioned medium induced Ser727 phosphorylation on Stat3 in hES cultures plated as aggregates in the absence of MEF cells. Overnight withdrawal of MEF CM resulted in almost complete loss of Ser727 phosphorylation, although STAT3 protein levels were not affected. JAK inhibition slightly reduced Ser727 phosphorylation levels. Tyr705 phosphorylation was absent in all conditions. FIG. 5c is a digital image showing JAK Inhibition caused a reduction in p38 phosphorylation levels in a time-dependent manner. FIG. 5d is a digital image showing p38 inhibition partly mimicked the effect of JAK inhibition on plating efficiency in hES cell cultures. FIG. 5e is a digital image showing p38 inhibition induced the de-phosphorylation of SMAD1/5/8 and MSK kinase, and the phosphorylation of STAT3 on Ser727. FIG. 5f is a digital image showing HSF6 hES cells cultured for 3 passages (3 weeks) with daily treatments of JAK inhibitor retained normal morphology and antigenic profile of undifferentiated hES cells. Scale bar: 300 μm. FIG. 5g is a digital image showing combined FGF-8 and sonic hedgehog treatment of hES cells generated TH+/TUJ1+ dopaminergic neurons. Scale bar: 300 μm. (Data in graphs represent means ±SD).

FIGS. 6a-h is a set of images showing Notch activation increased the generation of adult CNS stem cells and promoted behavioral recovery. FIG. 6a is a digital image showing immunohistochemical detection of BrdU/nestin in the SVZ of FGF2 and FGF2/Delta-4 treated animals at 1 week post-op (Scale bars: 200 μm). FIG. 6b is a digital image showing immunohistochemical detection of BrdU/DCX and BrdU/Sox2 in the SVZ of FGF2 and FGF2/Delta-4 treated animals at 1 week post-op (Scale bars: 50 μm). FIG. 6c is a bar graph showing quantification of BrdU at 7 days post-implantation in normal rats (Contralateral hemisphere). FIG. 6d is a bar graph showing quantification of BrdU at 45 days post-implantation in ischemic rats (Ipsilateral hemisphere). FIG. 6e is a digital image showing immunohistochemical detection of BrdU/DCX and BrdU/GFAP in the SVZ and DRdU/GFAP in the cerebral cortex of FGF2 and FGF2/Delta-4 treated animals at one week post-operatively (Scale bars: 50 μm). FIG. 6f is a digital image showing confocal images from rats treated with FGF2+Delta-4 and sacrificed at 45 days showed that approximately 30% of the BrdU+ cells in the cortex were also HU+, at 45 days post-implantation in stroked rats (Scale bars, 50 um). FIG. 6g is a bar graph showing quantification of BrdU at 45 days post-implantation in stroked rats. FIG. 6h is a graph illustrating motor skills improvement at the indicated days. Data in graphs represent Means ±Standard Error of the Mean (SEMs). SEMs were calculated by standard error divided by the sequence root of the number (N), where N=the number of independent experiments performed.

FIG. 7 is a schematic representation of signaling pathways involved in self-renewal and differentiation of stem cells.

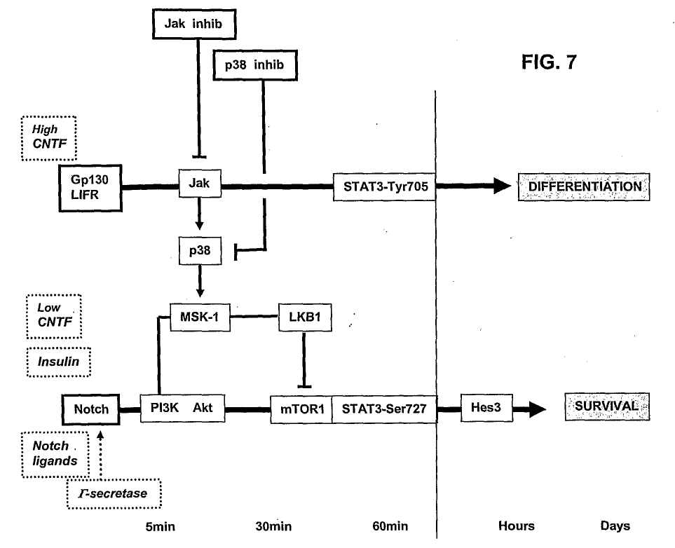

FIGS. 8a-8c are digital images showing ligand-induced Notch activation does not promote differentiation or commitment in neural stem cell cultures. FIG. 8a is a digital image of E13.5 neural stem cells treated for 5 days with either Jagged-1 or Delta-4 in the presence of FGF2 retained normal morphology, nestin expression and did not express differentiation markers (GFAP, TUJ1, CNPase) (“Undifferentiated”). Cells subjected to Notch activation for 5 days followed by Notch ligand and FGF2 withdrawal for four days gave rise to neurons (TUJ1), astrocytes (GFAP), and oligodendrocytes (CNPase). Blue staining: DAPI. Scale bars, 100 um. FIG. 8b is a digital image of triple staining of an E13.5 neural stem cell clone treated with FGF2 and Delta-4 for 7 days and allowed to differentiate by withdrawal of FGF2 and Delta-4 gave rise to neurons, astrocytes, and oligodendrocytes. Scale bars, 300 μm. FIG. 8c is a digital image showing CNS SC cultures from adult SVZ gave rise to neurons (TUJ1), astrocytes (GFAP), and oligodendrocytes (CNPase) following a 5 day expansion in FGF2 and a 7 day mitogen withdrawal period. Scale bars, 300 μm.

FIGS. 9A-9B are two digital images showing Hes3 mRNA responses to treatments with Notch ligands and CNTF. FIG. 9A is a digital image showing Jagged-induced elevation of Hes3 mRNA was sensitive to the γ-secretase inhibitor DAPT. CNTF (20 ng/ml) did not induce Hes3 mRNA. FIG. 9b is a digital image showing simultaneous CNTF and JAK Inhibitor treatment induced Hes3 mRNA.

FIGS. 10a-10c are a set of images showing JAK inhibition reduces the phosphorylation of STAT3 on Ser727 in mES cell cultures. FIG. 10a is a bar graph showing that in the presence of LIF, Delta-4 treatment and p38 inhibition did not significantly alter cell survival in a plating efficiency assay, whereas JAK inhibition reduced it. FIG. 10b is a digital image showing that at 90 min JAK inhibition caused a significant down-regulation in STAT3 phosphorylation on both Ser727 and Tyr705. Very low levels of phosphorylated p38 were detectable under all conditions. FIG. 10c is a digital image showing that five day treatments of mES cells with Delta-4 or p38 Inhibitor in the presence of LIF did not affect Oct4 expression, whereas JAK inhibition down-regulated Oct4. Self-renewal of mES cells correlated with Ser727 phosphorylation.

FIGS. 11a-11b are digital images of HSF6 hES cells cultured for 3 passages (3 weeks) with daily treatments of JAK inhibitor. FIG. 11a is a digital image showing the cells retained normal morphology and expression of Oct4, Tra-1-60, and Tra-1-81. FIG. 11b is a digital image showing Sox1/Nestin double-positive cell generation in embryoid bodies in the presence of serum-containing culture medium.

FIG. 12a-12b is a bar graph and a digital image showing increased generation of pancreatic precursors by Notch activation and inhibition of JAK and p38 kinases. FIG. 12a is a bar graph showing Notch activation and inhibition of JAK or p38 kinases increased the generation of pancreatic precursor aggregates in culture (Rat E14.5). FIG. 12b is a digital image showing these aggregates expressed markers of early pancreatic islets (HNF3β⁺/glucagon⁺/c-peptide⁺/somatostatin⁺).

### DETAILED DESCRIPTION

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided:

Agent: Any polypeptide, compound, small molecule, organic compound, salt, polynucleotide, or other molecule of interest.

Akt (protein kinase B): A serine/threonine kinase that is an enzyme involved in signal transduction pathways in cell proliferation, apoptosis, angiogenesis, and diabetes. In mammals three isoforms of Akt (a, b, g or Akt 1, 2, 3) have been described. These isoforms exhibit a high degree of homology, but differ slightly in the localization of their regulatory phosphorylation sites. Akta is the predominant isoform in most tissues, whereas the highest expression of Aktb is observed in the insulin-responsive tissues, and Aktg is abundant in brain tissue. Each Akt isoform is composed of three functionally distinct regions: an N-terminal pleckstrin homology (PH) domain that provides a lipid-binding module to direct Akt to phosphatidylinositol (PIP)₂ and PIP₃, a central catalytic domain, and a C-terminal hydrophobic motif.

Akt is constitutively phosphorylated at Ser¹²⁴, in the region between the PH and catalytic domains, and on Thr⁴⁵⁰, in the C-terminal region (in Akta, the most widely studied isoform) in unstimulated cells. Activation of Akt involves growth factor binding to a receptor tyrosine kinase and activation of PI 3-K, which phosphorylates membrane bound PIP₂ to generate PIP₃. The binding of PIP₃ to the PH domain anchors Akt to the plasma membrane and allows its phosphorylation and activation by PDK1. Akt is fully activated following its phosphorylation at two regulatory residues, a threonine residue on the kinase domain and a serine residue on the hydrophobic motif, which are structurally and functionally conserved within the AGC kinase family. Phosphorylation at Thr³⁰⁸ and Ser⁴⁷³ is required for the activation of Akta, while phosphorylation at Thr³⁰⁹ and Ser⁴⁷⁴ activates Aktb. Phosphorylation at Thr³⁰⁵ activates Aktg. Phosphorylation of a threonine residue on the kinase domain, catalyzed by PDK1, is essential for Akt activation. It causes a charge-induced conformational change, allowing substrate binding and increased rate of catalysis. Akt activity is augmented about 10-fold by phosphorylation at the serine residue by PDK2.

Alter: A change in an effective amount of a substance of interest, such as a polynucleotide or polypeptide. The amount of the substance can changed by a difference in the amount of the substance produced, by a difference in the amount of the substance that has a desired function, or by a difference in the activation of the substance. The change can be an increase or a decrease. The alteration can be in vivo or in vitro. In several embodiments, altering an effective amount of a polypeptide or polynucleotide is at least about a 50%, 60%, 70%, 80%, 90%, 95%, 96%, 97%, 98%, 99%, or 100% increase or decrease in the effective amount (level) of a substance, the proliferation and/or survival of a cells, or the activity of a proteins such as an enzyme. In another embodiment, an alteration in polypeptide or polynucleotide or enzymatic activity affects a physiological property of a cell, such as the differentiation, proliferation, or senescence of the cell.

Animal: Living multi-cellular vertebrate organisms, a category that includes, for example, mammals and birds. The term mammal includes both human and non-human mammals. Similarly, the term “subject” includes both human and veterinary subjects.

cDNA (complementary DNA): A piece of DNA lacking internal, non-coding segments (introns) and regulatory sequences that determine transcription. cDNA is synthesized in the laboratory by reverse transcription from messenger RNA extracted from cells.

Central Nervous System (CNS): The part of the nervous system of an animal that contains a high concentration of cell bodies and synapses and is the main site of integration of nervous activity. In higher animals, the CNS generally refers to the brain and spinal cord.

Cillary Neurotropic Factor (CNTF): An acidic cytosolic protein of approximately 24 kDa. CNTF does not display any homology to other neurotropic factors. At the protein level CNTF from rabbits and humans show approximately 76 percent sequence identity. Rat CNTF and human CNTF show 84 percent homology.

CNTF is found predominantly in peripheral nerve tissues. The main source appears to be myelin-associated Schwann cells in peripheral nerves and astrocytes in the central nervous system. CNTF appears to be expressed relatively late during ontogenesis. CNTF has been proposed to be a lesion factor that is released after nerve injuries and that, in combination with other factors, promotes the survival and the regeneration of neurons. In vitro CNTF promotes the growth of parasympathetic neurons and sympathetic, sensory, and spinal motor neurons.

Degenerate variant: A polynucleotide encoding a nucleostemin that includes a sequence that is degenerate as a result of the genetic code. There are 20 natural amino acids, most of which are specified by more than one codon. Therefore, all degenerate nucleotide sequences are included in the invention as long as the amino acid sequence of the nucleostemin polypeptide encoded by the nucleotide sequence is unchanged.

Differentiation: Refers to the process whereby relatively unspecialized cells (e.g., embryonic cells) acquire specialized structural and/or functional features characteristic of mature cells. Similarly, “differentiate” refers to this process. Typically, during differentiation, cellular structure alters and tissue-specific proteins appear.

Effective amount or Therapeutically effective amount: The amount of agent sufficient to prevent, treat, reduce and/or ameliorate the symptoms and/or underlying causes of any of a disorder or disease. In one embodiment, an “effective amount” is sufficient to reduce or eliminate a symptom of a disease. In another embodiment, an effective amount is an amount sufficient to overcome the disease itself.

Embryoid bodies: Embryonic stem (ES) cell aggregates generated when ES cells are plated on a non-adhesive surface that prevents attachment and differentiation of the ES cells. Generally, embryoid bodies include an inner core of undifferentiated stem cells surrounded by primitive endoderm.

Embryonic stem (ES) cells: Pluripotent cells isolated from the inner cell mass of the developing blastocyst. “ES cells” can be derived from any organism. ES cells can be derived from mammals. In one embodiment, ES cells are produced from mice, rats, rabbits, guinea pigs, goats, pigs, cows and humans. Human and murine derived ES cells are preferred. ES cells are totipotent cells, meaning that they can generate all of the cells present in the body (bone, muscle, brain cells, etc.). Methods for producing murine ES cells can be found in U.S. Pat. No. 5,670,372, herein incorporated by reference. Methods for producing human ES cells can be found, for example, in U.S. Pat. No. 6,090,622, PCT Publication No. WO 00/70021 and PCT Publication No. WO 00/27995, which are all herein incorporated by reference.

Epitope: An antigenic determinant. These are particular chemical groups or peptide sequences on a molecule that are antigenic, i.e. that elicit a specific immune response. An antibody specifically binds a particular antigenic epitope on a polypeptide.

Expand: A process by which the number or amount of cells in a cell culture is increased due to cell division. Similarly, the terms “expansion” or “expanded” refers to this process. The terms “proliferate,” “proliferation” or “proliferated” may be used interchangeably with the words “expand,” “expansion”, or “expanded.” Typically, during an expansion phase, the cells do not differentiate to form mature cells, but divide to form more cells.

Feeder layer: Non-proliferating cells (e.g. irradiated cells) that can be used to support proliferation of cells, including cells obtained from diverse sources including normal as well as neoplastic tissues from humans and laboratory animals. Protocols for the production of feeder layers are known in the art, and are available on the internet, such as at the National Stem Cell Resource website, which is maintained by the American Type Culture Collection (ATCC).

Fibroblast growth factor or FGF: Any suitable fibroblast growth factor, derived from any animal, and functional fragments thereof. A variety of FGFs are known and include, but are not limited to, FGF-1 (acidic fibroblast growth factor), FGF-2 (basic fibroblast growth factor, bFGF), FGF-3 (int-2), FGF-4 (hst/K-FGF), FGF-5, FGF-6, FGF-7, FGF-8, FGF-9 and FGF-98. “FGF” refers to a fibroblast growth factor protein such as FGF-1, FGF-2, FGF-4, FGF-6, FGF-8, FGF-9 or FGF-98, or a biologically active fragment or mutant thereof. The FGF can be from any animal species. In one embodiment, the FGF is mammalian FGF, including but not limited to, rodent, avian, canine, bovine, porcine, equine and human. The amino acid sequences and method for making many of the FGFs are well known in the art.

The amino acid sequence of human FGF-1 and a method for its recombinant expression are disclosed in U.S. Pat. No. 5,604,293. The amino acid sequence of human FGF-2 and methods for its recombinant expression are disclosed in U.S. Pat. No. 5,439,818, herein incorporated by reference. The amino acid sequence of bovine FGF-2 and various methods for its recombinant expression are disclosed in U.S. Pat. No. 5,155,214, also herein incorporated by reference. When the 146 residue forms are compared, their amino acid sequences are nearly identical, with only two residues that differ.

The amino acid sequence of FGF-3 (Dickson et al., Nature 326:833, 1987) and human FGF-4 (Yoshida et al., PHAS USA 84:7305-7309, 1987) are known. When the amino acid sequences of human FGF-4, FGF-1, FGF-2 and murine FGF-3 are compared, residues 72-204 of human FGF-4 have 43% homology to human FGF-2; residues 79-204 have 38% homology to human FGF-1; and residues 72-174 have 40% homology to murine FGF-3. The cDNA and deduced amino acid sequences for human FGF-5 (Zhan et al., Molec. and Cell. Biol. 8(8):3487-3495, 1988), human FGF-6 (Coulier et al., Oncogene 6:1437-1444, 1991), human FGF-7 (Miyamoto et al., Mol. and Cell. Biol. 13(7):4251-4259, 1993) are also known. The cDNA and deduced amino acid sequence of murine FGRF-8 (Tanaka et al., PNAS USA 89:8928-8932, 1992), human and murine FGF-9 (Santos-Ocamp et al., J. Biol. Chem. 271(3):1726-1731, 1996) and human FGF-98 (provisional patent application Ser. No. 60/083,553, which is hereby incorporated herein by reference in its entirety) are also known.

FGF-2 (also known as bFGF or bFGF-2), and other FGFs, can be made as described in U.S. Pat. No. 5,155,214 (“the '214 patent”). The recombinant bFGF-2, and other FGFs, can be purified to pharmaceutical quality (98% or greater purity) using the techniques described in detail in U.S. Pat. No. 4,956,455.

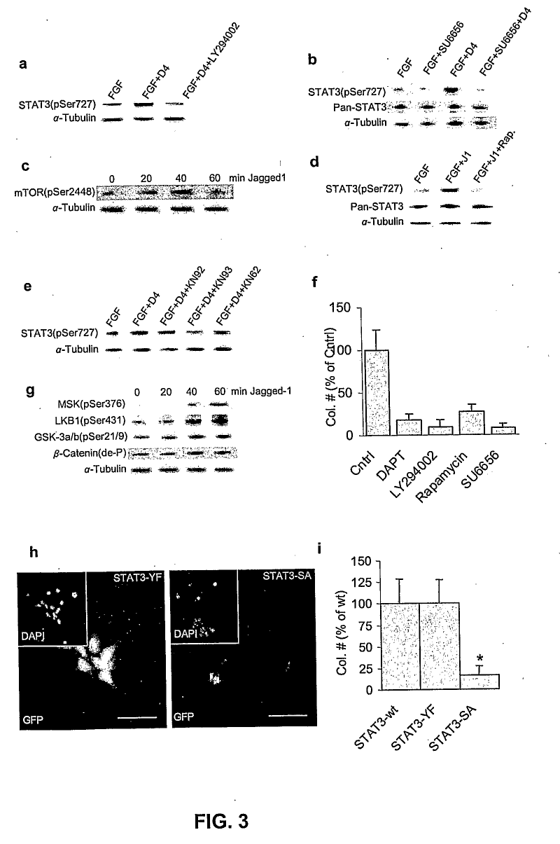

FGF-4 is the product of the hst oncogene (also known as hst-1 or hst). The amino acid sequence for human FGF-4 was first disclosed by Yoshida et al., Proc. Natl. Acad. Sci. USA 84:7305-7309, 1987, at FIG. 3. The endogenous human protein encoded has a molecular mass of 23 kDa. FGF-4 has been implicated recently as one of the molecules that directs outgrowth and patterning of the limb during chick embryonic growth (see Adelaide et al., Oncogene 2:413-416, 1988; see also U.S. Pat. No. 6,277,820).

Fibroblast growth factor-8 (FGF-8), alternatively known as androgen-induced growth factor (AIGF) is a member of the FGF family known to influence embryogenesis and morphogenesis. The in situ embryonic expression pattern suggests a unique role of FGF-8 in mouse development, especially in gastrulation, brain development, and limb and facial morphogenesis. Ohuchi et al., Biochem. Biophys. Res. Commun. 204(2):882-888, 1994. Northern blot expression reveals a unique temporal and spatial pattern of FGF-8 expression in the developing mouse and suggests a role for this FGF in multiple regions of ectodermal differentiation in the post-gastrulation mouse embryo. Heikinheimo et al., Mech. Dev. 48(2): 129-138, 1994. A sequence of FGF-8 is shown in U.S. Pat. No. 6,277,820.

Biologically active variants of FGF are also of use with the methods disclosed herein. Such variants should retain FGF activities, particularly the ability to bind to FGF receptor sites. FGF activity may be measured using standard FGF bioassays, which are known to those of skill in the art. Representative assays include known radioreceptor assays using membranes, a bioassay that measures the ability of the molecule to enhance incorporation of tritiated thymidine, in a dose-dependent manner, into the DNA of cells, and the like. Preferably, the variant has at least the same activity as the native molecule.

In addition to the above described FGFs, an agent of use also includes an active fragment of any one of the above-described FGFs. In its simplest form, the active fragment is made by the removal of the N-terminal methionine, using well-known techniques for N-terminal methionine removal, such as a treatment with a methionine aminopeptidase. A second desirable truncation includes an FGF without its leader sequence. Those skilled in the art recognize the leader sequence as the series of hydrophobic residues at the N-terminus of a protein that facilitate its passage through a cell membrane but that are not necessary for activity and that are not found on the mature protein.

Preferred truncations on the FGFs are determined relative to mature FGF-2 having 146 residues. As a general rule, the amino acid sequence of an FGF is aligned with FGF-2 to obtain maximum homology. Portions of the FGF that extend beyond the corresponding N-terminus of the aligned FGF-2 are generally suitable for deletion without adverse effect. Likewise, portions of the FGF that extend beyond the C-terminus of the aligned FGF-2 are also capable of being deleted without adverse effect.

Fragments of FGF that are smaller than those described can also be employed in the present methods. It should be noted that human and murine FGF-2, FGF-4, FGF-8 and a variety of other FGFs, are commercially available.

Suitable biologically active variants can be FGF analogs or derivatives. By “analog” is intended an analog of either FGF or an FGF fragment that includes a native FGF sequence and structure having one or more amino acid substitutions, insertions, or deletions. Analogs having one or more peptoid sequences (peptide mimic sequences) are also included (see e.g. International Publication No. WO 91/04282). By “derivative” is intended any suitable modification of FGF, FGF fragments, or their respective analogs, such as glycosylation, phosphorylation, or other addition of foreign moieties, as long as the FGF activity is retained. Methods for making FGF fragments, analogs and derivatives are available in the art.

In addition to the above-described FGFs, the methods disclosed herein can also employ an active mutant or variant thereof. By the term active mutant, as used in conjunction with an FGF, is meant a mutated form of the naturally occurring FGF. FGF mutant or variants will generally have at least 70%, preferably 80%, more preferably 85%, even more preferably 90% to 95% or more, and for example 98% or more amino acid sequence identity to the amino acid sequence of the reference FGF molecule. A mutant or variant may, for example, differ by as few as 1 to 10 amino acid residues, such as 6-10, as few as 5, as few as 4, 3, 2, or even 1 amino acid residue.

The sequence identity can be determined as described herein. For FGF, one method for determining sequence identify employs the Smith-Waterman homology search algorithm (Meth. Mol. Biol. 70:173-187, 1997) as implemented in MSPRCH program (Oxford Molecular) using an affine gap search with the following search parameters: gap open penalty of 12, and gap extension penalty of 1. In one embodiment, the mutations are “conservative amino acid substitutions” using L-amino acids, wherein one amino acid is replaced by another biologically similar amino acid. Conservative amino acid substitutions are those that preserve the general charge, hydrophobicity, hydrophilicity, and/or steric bulk of the amino acid being substituted.

One skilled in the art, using art known techniques, is able to make one or more point mutations in the DNA encoding any of the FGFs to obtain expression of an FGF polypeptide mutant (or fragment mutant) having an activity for use in methods disclosed herein. To prepare a biologically active mutant of an FGF, one uses standard techniques for site directed mutagenesis, as known in the art and/or as taught in Gilman et al., Gene 8:81, 1979 or Roberts et al., Nature 328:731, 1987, to introduce one or more point mutations into the cDNA that encodes the FGF.

Growth factor: A substance that promotes cell growth, survival, and/or differentiation. Growth factors include molecules that function as growth stimulators (mitogens), molecules that function as growth inhibitors (e.g. negative growth factors) factors that stimulate cell migration, factors that function as chemotactic agents or inhibit cell migration or invasion of tumor cells, factors that modulate differentiated functions of cells, factors involved in apoptosis, or factors that promote survival of cells without influencing growth and differentiation. Examples of growth factors are a fibroblast growth factor (such as FGF-2), epidermal growth factor (EGF), cilliary neurotrophic factor (CNTF), and nerve growth factor (NGF), and actvin-A. In one specific, non-limiting example, a growth factor is insulin.

Growth medium or expansion medium: A synthetic set of culture conditions with the nutrients necessary to support the growth (cell proliferation/expansion) of a specific population of cells. In one embodiment, the cells are stem cells, such as ES cells or neuronal stem cells. Growth media generally include a carbon source, a nitrogen source and a buffer to maintain pH. In one embodiment, ES growth medium contains a minimal essential media, such as DMEM, supplemented with various nutrients to enhance ES cell growth. Additionally, the minimal essential media may be supplemented with additives such as horse, calf or fetal bovine serum.

Hairy and Enhancer of Split3 (Hes3): The Hes gene family members are mammalian homologues of the Drosophila hairy and Enhancer of split genes. Hairy and Enhancer of Split function in both segmentation and in the Notch neurogenic pathway during Drosophila embryo development. A conserved role for the Hes genes is in the Notch signaling pathway. During early development of the central nervous system, Hes3 is expressed in the region of the midbrain/hindbrain boundary, and in rhombomeres 2, 4, 6 and 7. Later in development, Hes3 is co-expressed with other neurogenic gene homologues in the developing central nervous system and epithelial cells undergoing mesenchyme induction. An exemplary human Hes3 sequence is set forth as GENBANK® Accession No. NM_{—}001024598 and an exemplary murine Hes3 sequence is set forth s GENBANK® Accession No. NM_{—}008237, both of which are incorporated by reference herein in their entirety.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Hybridization: A process wherein oligonucleotides and their analogs bind by hydrogen bonding, which includes Watson-Crick, Hoogsteen or reversed Hoogsteen hydrogen bonding, between complementary bases. Generally, nucleic acid consists of nitrogenous bases that are either pyrimidines (Cytosine (C), uracil (U), and thymine(T)) or purines (adenine (A) and guanine (G)). These nitrogenous bases form hydrogen bonds consisting of a pyrimidine bonded to a purine, and the bonding of the pyrimidine to the purine is referred to as “base pairing.” More specifically, A will bond to T or U, and G will bond to C. “Complementary” refers to the base pairing that occurs between two distinct nucleic acid sequences or two distinct regions of the same nucleic acid sequence. In one embodiment, nucleic acids that encode growth factors or Notch can be used to produce these proteins. Sequences that hybridize to these nucleic acid molecules, that produce functional proteins, can also be used to produce Notch ligands or growth factors.

“Specifically hybridizable” and “specifically complementary” are terms which indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions in which specific binding is desired, for example under physiological conditions in the case of in vivo assays. Such binding is referred to as “specific hybridization.”

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ concentration) of the hybridization buffer will determine the stringency of hybridization.

Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC or SSPE). Then, assuming that 1% mismatching results in a 1° C. decrease in the Tm, the temperature of the final wash in the hybridization reaction is reduced accordingly (for example, if sequences having >95% identity with the probe are sought, the final wash temperature is decreased by 5° C.). In practice, the change in Tm can be between 0.5° C. and 1.5° C. per 1% mismatch. The parameters of salt concentration and temperature can be varied to achieve the optimal level of identity between the probe and the target nucleic acid. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11, herein incorporated by reference.

For purposes of this disclosure, “stringent conditions” encompass conditions under which hybridization will only occur if there is less than 30% mismatch between the hybridization molecule and the target sequence. “Stringent conditions” may be broken down into particular levels of stringency for more precise definition. Thus, as used herein, “moderate stringency” conditions are those under which molecules with more than 30% sequence mismatch will not hybridize; conditions of “medium stringency” are those under which molecules with more than 20% mismatch will not hybridize, and conditions of “high stringency” are those under which sequences with more than 10% mismatch will not hybridize.

Isolated: An “isolated” biological component (such as a nucleic acid, peptide or protein) has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids. Similarly, an “isolated” cell has been substantially separated, produced apart from, or purified away from other cells of the organism in which the cell naturally occurs. Isolated cells can be, for example, at least 99%, at least 98%, at least 95%, at least 90%, at least 85%, or at least 80% pure.

Janus Activated Kinase (JAK)/Signal Transducer and Activator of Transcription (STAT): JAKs are cytoplasmic tyrosine kinases that are either constitutively associated with cytokine receptors or recruited to receptors after ligand binding. In either case, stimulation with the ligand results in the catalytic activation of receptor-associated JAKs. This activation results in the phosphorylation of cellular substrates, including the JAK-associated cytokine receptor chains. Some of these phosphorylated tyrosines can serve as coding sites for STAT proteins, which bind to the phosphotyrosines by their SRC-homology 2 (SH2) domains. STAT proteins are also phosphorylated on a conserved tyrosine residue (tyrosine 705 in STAT3), resulting in their dimerization and acquisition of high-affinity DNA-binding activity, which facilitates their action as nuclear transcription factors.

STAT3 is a major cell signaling constituent with roles in both survival and differentiation. However, STAT3 can be phosphorylated on two major residues, Tyrosine (Tyr)705 and Serine (Ser)727. Tyr705 phosphorylation is mediated by JAK2 and Src kinases. Ser727 phosphorylation is mediated by ERK, JNK kinases, TAK1-NLK kinases, and mTOR. Akt and mTOR are also known to mediate survival and growth in many cell types.

The JAK/STAT pathway is one of the most rapid cytoplasmic to nuclear signaling mechanisms. There are a total of four JAK (JAK1-3 and tyrosine kinase 2) and seven STAT proteins (STAT1-4, STAT5A, STAT5b and STAT6). JAKs are relatively large cytoplasmic kinases of about 1,100 amino acids in length, and range in size from about 116 kDa to about 140 kDa. The STAT proteins can dimerize, translocate to the nucleus, and bind DNA. Binding of the STAT proteins to the DNA can result in the activation of transcription (for review see Leonard, Nature Reviews 1: 200-208, 2001).

“STAT inhibitor,” “JAK inhibitor,” and “JAK/STAT inhibitor” are used to refer to any agent capable of down-regulating or otherwise decreasing or suppressing the amount and/or activity of JAK-STAT interactions. JAK inhibitors down-regulate the quantity or activity of JAK molecules. STAT inhibitors down-regulate the quantity or activity of STAT molecules. Inhibition of these cellular components can be achieved by a variety of mechanisms known in the art, including, but not limited to binding directly to JAK (for example, a JAK-inhibitor compound binding complex, or substrate mimetic), binding directly to STAT, or inhibiting the expression of the gene, which encodes the cellular components. JAK/STAT inhibitors are disclosed in U.S. Patent Publication No. 2004/0209799).

Kinase: An enzyme that catalyzes the transfer of a phosphate group from one molecule to another. Kinases play a role in the regulation of cell proliferation, differentiation, metabolism, migration, and survival. A “serine threonine kinase” transfers phosphate groups to a hydroxyl group of serine and/or threonine in a polypeptide.

Receptor protein tyrosine kinases (PTKs) contain a single polypeptide chain with a transmembrane segment. The extracellular end of this segment contains a high affinity ligand-binding domain, while the cytoplasmic end comprises the catalytic core and the regulatory sequences. The cytosolic end also contains tyrosine residues, which become substrates or targets for the tyrosine kinase portion of the receptor. PTK remains inactive until a ligand binds to the receptor, which leads to the dimerization of two ligand-bound receptors (exception: insulin receptor). Once activated, receptors are able to autophosphorylate tyrosine residues outside the catalytic domain. This stabilizes the active receptor conformation and creates phosphotyrosine-docking sites for proteins that transduce signals within the cell.

The cytosolic portion of the phosphorylated receptor recruits a number of cytosolic adapter proteins via interactions between phosphorylated tyrosine residues on the receptor and the SH2 domain on the adapter molecule. Different proteins have different SH2 domains that recognize specific phosphotyrosine residues. An SH2-containing protein, Grb2, acts as a common adapter protein in a majority of growth factor related signaling events.

Non-receptor tyrosine kinases include members of the Src, Tec, JAK, Fes, Abl, FAK, Csk, and Syk families. They are located in the cytoplasm as well as in the nucleus. They exhibit distinct kinase regulation, substrate phosphorylation, and function. In most cases, their activation also begins with the phosphorylation of a tyrosine residue present in an activation loop.

One example of a kinase is a JAK (see above). Another example of a kinase is a “phosphatidyl inositol 3-kinase,” an enzyme that phosphorylates inositol lipids at the D-3 position of the inositol ring to generate the 3-phosphoinositides, phosphatidylinositol 3-phosphate [PtdIns(3)P], phosphatidylinositol 3,4-bisphosphate [PtdIns(3,4)P₂] and phosphatidylinositol 3,4,5-trisphosphate [PtdIns(3,4,5)P₃]. GENBANK® Accession No. AAB53966 (May 9, 1997) sets forth an exemplary amino acid sequence of the catalytic subunit of human phosphatidyl inositol 3-kinase. A “preferential” inhibition of a kinase refers to decreasing activity of one kinase, such as MAP kinase (see below), more than inhibiting the activity of a second kinase, such as JAK.

Mitogen-activated protein kinases (MAP Kinases): A group of protein serine/threonine kinases that are activated in response to a variety of extracellular stimuli and mediate signal transduction from the cell surface to the nucleus. In combination with several other signaling pathways, they can differentially alter phosphorylation status of the transcription factors. A controlled regulation of these cascades is involved in cell proliferation and differentiation.

The p38 kinase (“p38”) is the most well-characterized member of the MAP kinase family. It is activated in response to inflammatory cytokines, endotoxins, and osmotic stress. It shares about 50% homology with the ERKs. However, downstream activation of p38 occurs following its phosphorylation (at the TGY motif) by MKK3, a dual specificity kinase. Following its activation, p38 translocates to the nucleus and phosphorylates ATF-2.

Neurological disorder: A disorder in the nervous system, including the central nervous system (CNS) and peripheral nervous system (PNS). Examples of neurological disorders include Parkinson's disease, Huntington's disease, Alzheimer's disease, severe seizure disorders including epilepsy, familial dysautonomia as well as injury or trauma to the nervous system, such as neurotoxic injury or disorders of mood and behavior such as addiction, schizophrenia and amyotrophic lateral sclerosis. Neuronal disorders also include Lewy body dementia, multiple sclerosis, epilepsy, cerebellar ataxia, progressive supranuclear palsy, amyotrophic lateral sclerosis, affective disorders, anxiety disorders, obsessive compulsive disorders, personality disorders, attention deficit disorder, attention deficit hyperactivity disorder, Tourette Syndrome, Tay Sachs, Nieman Pick, and other lipid storage and genetic brain diseases and/or schizophrenia.

Neurodegenerative disorder: An abnormality in the nervous system of a subject, such as a mammal, in which neuronal integrity is threatened. Without being bound by theory, neuronal integrity can be threatened when neuronal cells display decreased survival or when the neurons can no longer propagate a signal. Specific, non-limiting examples of a neurodegenerative disorder are Alzheimer's disease, Pantothenate kinase associated neurodegeneration, Parkinson's disease, Huntington's disease (Dexter et al., Brain 114:1953-1975, 1991), HIV encephalopathy (Miszkziel et al., Magnetic Res. Imag. 15:1113-1119, 1997), and amyotrophic lateral sclerosis.

Alzheimer's disease manifests itself as pre-senile dementia. The disease is characterized by confusion, memory failure, disorientation, restlessness, speech disturbances, and hallucination in mammals (Medical, Nursing, and Allied Health Dictionary, 4th Ed., 1994, Editors: Anderson, Anderson, Glanze, St. Louis, Mosby). Alzheimer's disease is characterized by a progressive loss of neurons, formation of fibrillary tangles within neurons and numerous plaques in affected brain regions. It is believed that the key pathogenic event in Alzheimer's disease is the excessive formation and/or accumulation of fibrillar β-amyloid peptides, which are also called αβ.

Parkinson's disease is a slowly progressive, degenerative, neurologic disorder characterized by resting tremor, loss of postural reflexes, and muscle rigidity and weakness (Medical, Nursing, and Allied Health Dictionary, 4th Ed., 1994, Editors: Anderson, Anderson, Glanze, St. Louis, Mosby).

Amyotrophic lateral sclerosis is a degenerative disease of the motor neurons characterized by weakness and atrophy of the muscles of the hands, forearms and legs, spreading to involve most of the body and face (Medical, Nursing, and Allied Health Dictionary, 4th Ed., 1994, Editors: Anderson, Anderson, Glanze, St. Louis, Mosby).

Pantothenate kinase associated neurodegeneration (PKAN, also known as Hallervorden-Spatz syndrome) is an autosomal recessive neurodegenerative disorder associated with brain iron accumulation. Clinical features include extrapyramidal dysfunction, onset in childhood, and a relentlessly progressive course (Dooling et al., Arch. Neurol. 30:70-83, 1974). PKAN is a clinically heterogeneous group of disorders that includes classical disease with onset in the first two decades, dystonia, high globus pallidus iron with a characteristic radiographic appearance (Angelini et al., J. Neurol. 239:417-425, 1992), and often either pigmentary retinopathy or optic atrophy (Dooling et al., Arch. Neurol. 30:70-83, 1974; Swaiman et al., Arch. Neurol 48:1285-1293, 1991).

A “neurodegenerative related disorder” is a disorder such as speech disorders that are associated with a neurodegenerative disorder. Specific non-limiting examples of a neurodegenerative related disorders include, but are not limited to, palilalia, tachylalia, echolalia, gait disturbance, preservative movements, bradykinesia, spasticity, rigidity, retinopathy, optic atrophy, dysarthria, and dementia.

Nestin: A protein whose expression distinguishes neural multi-potential stem cells and brain tumor cells from the more differentiated neural cell types (such as neuronal, glial and muscle cells) of the mammalian brain. Nestin is an intermediate filament. The similarity between the nestin gene and the genes of the other five classes of intermediate filaments ranges from 16% to 29% at the amino acid level in a 307 amino acid long region starting close to the N-terminus of the nestin gene, corresponding to the conserved alpha-helical rod or “core” domain of the intermediate filaments. This region of the predicted nestin amino acid sequence also contains a repeated hydrophobic heptad motif characteristic of intermediate filaments. Amino acid sequences of nestin are disclosed, for example, in U.S. Pat. No. 5,338,839, which is incorporated herein by reference.

Notch: An integral membrane protein of 2703 amino acids that was first identified in Drosophila. Notch is the Drosophila homologue of the human epidermal growth factor (EGF) ceptor. Mammals have more than one Notch gene homolog. The Notch-1 gene is located human chromosome 9q34; the structure of Notch-1 is similar to Notch-2 (found on human chromosome 1p13-p11). Notch-3 (found on human chromosome 19p13.2-p13.1) lacks some of the domains found in the other family members and encodes a considerably shorter intracellular domain.

The intracellular domain of Notch has a length of approximately 1000 amino acids and is composed of a number of different sequence domains. The extracellular domain of wild-type Notch contains 36 EGF-like repeats that differ slightly in sequence. Some of these repeats are involved in the dimerisation and multimerisation of the Notch protein. Other repeats function as receptor domains for proteins involved in the differentiation of cells into neural and epidermal precursors.

Exemplary Notch amino acid sequences are as follows:

| Notch Protein | GENBANK ® Accession No.¹ |
|---|---|
| Human Notch 1 | NM_017617 (Sep. 3, 2006) |
| Human Notch 2 | NM_024408 (Aug. 28, 2006) |
| Human Notch 3 | NM_000435 (Sep. 3, 2006) |
| Human Notch 4 | NM_004557 (Aug. 28, 2006) |
| Mouse Notch 1 | NM_008714 (Aug. 20, 2006) |
| Mouse Notch 2 | NM_010928 (Aug. 6, 2006) |
| Mouse Notch 3 | NM_008716 (Aug. 6, 2006) |
| Mouse Notch 4 | NM_010929 (Aug. 6, 2006) |

¹All GENBANK ® data is incorporated by refernece herein. Dates expressed as month-day-year.

Two of the 36 EGF-repeats of the extracellular domain of Notch interact with another protein, called Delta and with other proteins, Serrate, and Lag-2, These proteins are collectively referred to also Notch ligands or DSL ligands. Jagged (also called Serrate-1) is also a Notch ligand. (see Artavanis-Tsakonas et al., Annual Review of Cell Biology 7: 427-452, 1991; U.S. Pat. No. 6,083,904, U.S. Pat. No. 6,149,902, and U.S. Pat. No. 5,780,3000, which are herein incorporated by reference. Delta proteins and nucleic acids are disclosed in U.S. Pat. No. 6,783,956, which is incorporated herein by reference.

Nucleostemin: A polypeptide that is involved in the controlling cell proliferation and differentiation, see PCT Publication No. WO 2004/031731 A2, which is incorporated herein by reference. The mouse, rat, and human nucleostemin polypeptides have been described.

Nucleotide: A monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

p75: A receptor, also called p75^{NTR}, that binds NGF and other neurotrophins and belongs to the family of death receptors. P75 has independent signaling capacity and mediates apoptosis, including the apoptosis induced by amyloid peptides. The cytoplasmic domain of p75 contains a putative death domain and a juxtamembrane intracellular domain.

Peripheral Nervous System (PNS): The part of an animal's nervous system other than the Central Nervous System. Generally, the PNS is located in the peripheral parts of the body and includes cranial nerves, spinal nerves and their branches, and the autonomic nervous system.

Polypeptide: A polymer in which the monomers are amino acid residues which are joined together through amide bonds. When the amino acids are alpha-amino acids, either the L-optical isomer or the D-optical isomer can be used, the L-isomers being preferred. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced.

The term “polypeptide fragment” refers to a portion of a polypeptide which exhibits at least one useful epitope. The term “functional fragments of a polypeptide” refers to all fragments of a polypeptide that retain an activity of the polypeptide, such as a nucleostemin. Biologically functional fragments, for example, can vary in size from a polypeptide fragment as small as an epitope capable of binding an antibody molecule to a large polypeptide capable of participating in the characteristic induction or programming of phenotypic changes within a cell, including affecting cell proliferation or differentiation. An “epitope” is a region of a polypeptide capable of binding an immunoglobulin generated in response to contact with an antigen. Thus, smaller peptides containing the biological activity of insulin, or conservative variants of the insulin, are thus included as being of use. The term “soluble” refers to a form of a polypeptide that is not inserted into a cell membrane.

The term “substantially purified polypeptide” as used herein refers to a polypeptide which is substantially free of other proteins, lipids, carbohydrates or other materials with which it is naturally associated. In one embodiment, the polypeptide is at least 50%, for example at least 80% free of other proteins, lipids, carbohydrates or other materials with which it is naturally associated. In another embodiment, the polypeptide is at least 90% free of other proteins, lipids, carbohydrates or other materials with which it is naturally associated. In yet another embodiment, the polypeptide is at least 95% free of other proteins, lipids, carbohydrates or other materials with which it is naturally associated.

Conservative substitutions (or “conservative variants”) replace one amino acid with another amino acid that is similar in size, hydrophobicity, etc. Examples of conservative substitutions are shown below.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Variations in the cDNA sequence that result in amino acid changes, whether conservative or not, should be minimized in order to preserve the functional and immunologic identity of the encoded protein. Thus, in several non-limiting examples, a nucleostemin polypeptide includes at most two, at most five, at most ten, at most twenty, or at most fifty conservative substitutions. The immunologic identity of the protein may be assessed by determining whether it is recognized by an antibody; a variant that is recognized by such an antibody is immunologically conserved. Any cDNA sequence variant will preferably introduce no more than twenty, and preferably fewer than ten amino acid substitutions into the encoded polypeptide. Variant amino acid sequences may, for example, be 80%, 90% or even 95% or 98% identical to the native amino acid sequence.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate.

Pharmaceutical agent: A chemical compound, small molecule, or other composition capable of inducing a desired therapeutic or prophylactic effect when properly administered to a subject or a cell. “Incubating” includes a sufficient amount of time for a drug to interact with a cell. “Contacting” includes incubating a drug in solid or in liquid form with a cell.

Polynucleotide: A nucleic acid sequence (such as a linear sequence) of any length. Therefore, a polynucleotide includes oligonucleotides, and also gene sequences found in chromosomes. An “oligonucleotide” is a plurality of joined nucleotides joined by native phosphodiester bonds. An oligonucleotide is a polynucleotide of between 6 and 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques. Similarly, a recombinant protein is one encoded by a recombinant nucleic acid molecule.

Senescence: The inability of a cell to divide further. A senescent cell is still viable, but does not divide.

Sequence identity: The similarity between amino acid sequences, such as growth factor or Notch ligand amino acid sequences, is expressed in terms of the percentage of conservation between the sequences, otherwise referred to as sequence similarity. Sequence identity is frequently measured in terms of percentage identity (or similarity or homology); the higher the percentage, the more similar the two sequences are. Homologues or variants of a growth factor or a Notch ligand will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet., 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the Internet, for use in connection with the sequence analysis programs blastp, blastn, blastx, tblastn and tblastx. A description of how to determine sequence identity using this program is available on the NCBI website on the Internet. Other specific, non-limiting examples of sequence alignment programs specifically designed to identify conserved regions of genomic DNA of greater than or equal to 100 nucleotides are PIPMaker (Schwartz et al., Genome Research 10: 577-586, 2000) and DOTTER (Erik et al., Gene 167: GC1-10, 1995).

Homologues and variants of a nucleic acid sequence are typically characterized by possession of at least 75%, for example at least 80%, 90%, 95%, 98%, or 99%, sequence identity counted over the full length alignment with the originating sequence using the NCBI Blast 2.0, set to default parameters. Methods for determining sequence identity over such short windows are available at the NCBI website on the Internet. One of skill in the art will appreciate that these sequence identity ranges are provided for guidance only; it is entirely possible that strongly significant homologues could be obtained that fall outside of the ranges provided.

Stem cell: A cell that can generate a fully differentiated functional cell of a more than one given cell type. The role of stem cells in vivo is to replace cells that are destroyed during the normal life of an animal. Generally, stem cells can divide without limit and are totipotent. After division, the stem cell may remain as a stem cell, become a precursor cell, or proceed to terminal differentiation. A central nervous system (CNS) stem cell is a cell of the central nervous system that can self-renew and can generate astrocytes, neurons and oligodendrocytes.

A “somatic precursor cell” is a cell that can generate a fully differentiated functional cell of at least one given cell type from the body of an animal, such as a human. A neuronal precursor cell can generate of fully differentiated neuronal cell, such as, but not limited to, and adrenergic or a cholinergic neuron. A glial precursor cell can generate fully differentiated glial cells, such as but not limited to astrocytes, microglia and oligodendroglia. Generally, precursor cells can divide and are pluripotent. After division, a precursor cell can remain a precursor cell, or may proceed to terminal differentiation. A neuronal precursor cell can give rise to one or more types of neurons, such as dopaminergic, adrenergic, or serotinergic cells, but is more limited in its ability to differentiate than a stem cell. In one example, a neuronal stem cell gives rise to all of the types of neuronal cells (such as dopaminergic, adrenergic, and serotinergic neurons) but does not give rise to other cells, such as glial cells.

Sonic hedgehog (SHH): Sonic hedgehog (SHH) is one of three mammalian homologs of the Drosophila hedgehog signaling molecule and is expressed at high levels in the notochord and floor plate of developing embryos. SHH is known to play a key role in neuronal tube patterning (Echerlard et al., Cell 75:1417-30, 1993), the development of limbs, somites, lungs and skin. Moreover, overexpression of SHH has been found in basal cell carcinoma. Exemplary amino acid sequences of SHH is set forth in U.S. Pat. No. 6,277,820.

Subject: Any mammal, such as humans, non-human primates, pigs, sheep, cows, rodents and the like, which is to be the recipient of the particular treatment. In one embodiment, a subject is a human subject or a murine subject.

Survival (of a Cell): The length of time a given cell is alive. An increase in survival following treatment indicates that the cell lives for a longer length of time as compared to a control, such as the cell in the absence of treatment.

Synapse: Highly specialized intercellular junctions between neurons and between neurons and effector cells across which a nerve impulse is conducted (synaptically active). Generally, the nerve impulse is conducted by the release from one neuron (presynaptic neuron) of a chemical transmitter (such as dopamine or serotonin) which diffuses across the narrow intercellular space to the other neuron or effector cell (post-synaptic neuron). Generally neurotransmitters mediate their effects by interacting with specific receptors incorporated in the post-synaptic cell. “Synaptically active” refers to cells (e.g., differentiated neurons) which receive and transmit action potentials characteristic of mature neurons.

Therapeutic agent: Used in a generic sense, it includes treating agents, prophylactic agents, and replacement agents.

Transduced and Transformed: A virus or vector “transduces” a cell when it transfers nucleic acid into the cell. A cell is “transformed” or “transfected” by a nucleic acid transduced into the cell when the DNA becomes stably replicated by the cell, either by incorporation of the nucleic acid into the cellular genome, or by episomal replication.

Numerous methods of transfection are known to those skilled in the art, such as: chemical methods (e.g., calcium-phosphate transfection), physical methods (e.g., electroporation, microinjection, particle bombardment), fusion (e.g., liposomes), receptor-mediated endocytosis (e.g., DNA-protein complexes, viral envelope/capsid-DNA complexes) and by biological infection by viruses such as recombinant viruses (Wolff, J. A., ed, Gene Therapeutics, Birkhauser, Boston, USA (1994)). In the case of infection by retroviruses, the infecting retrovirus particles are absorbed by the target cells, resulting in reverse transcription of the retroviral RNA genome and integration of the resulting provirus into the cellular DNA. Methods for the introduction of genes into the pancreatic endocrine cells are known (e.g. see U.S. Pat. No. 6,110,743, herein incorporated by reference). These methods can be used to transduce a pancreatic endocrine cell produced by the methods described herein, or an artificial islet produced by the methods described herein.

Genetic modification of the target cell is one indicia of successful transfection. “Genetically modified cells” refers to cells whose genotypes have been altered as a result of cellular uptakes of exogenous nucleotide sequence by transfection. A reference to a transfected cell or a genetically modified cell includes both the particular cell into which a vector or polynucleotide is introduced and progeny of that cell.

Transgene: An exogenous gene supplied by a vector.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Methods for Increasing the Survival and/or Proliferation of Stem and/or Somatic Precursor Cells

Methods are disclosed herein for increasing the survival and/or proliferation of mammalian stem cells or precursor cells, such as somatic precursor cells. The cells can be in vivo or in vitro.

The cells can be totipotent cells or pluripotent cells. In one example, the cells are stem cells, such as embryonic stem cells. For example, murine, primate or human cells can be utilized. ES cells can proliferate indefinitely in an undifferentiated state. Furthermore, ES cells are totipotent cells, meaning that they can generate all of the cells present in the body (bone, muscle, brain cells, etc.). ES cells have been isolated from the inner cell mass (ICM) of the developing murine blastocyst (Evans et al., Nature 292:154-156, 1981; Martin et al., Proc. Natl. Acad. Sci. 78:7634-7636, 1981; Robertson et al., Nature 323:445-448, 1986). Additionally, human cells with ES properties have been isolated from the inner blastocyst cell mass (Thomson et al., Science 282:1145-1147, 1998) and developing germ cells (Shamblott et al., Proc. Natl. Acad. Sci. USA 95:13726-13731, 1998), and human and non-human primate embryonic stem cells have been produced (see U.S. Pat. No. 6,200,806, which is incorporated by reference herein).

As disclosed in U.S. Pat. No. 6,200,806, ES cells can be produced from human and non-human primates. In one embodiment, primate ES cells are isolated “ES medium” that express SSEA-3; SSEA-4, TRA-1-60, and TRA-1-81 (see U.S. Pat. No. 6,200,806). ES medium consists of 80% Dulbecco's modified Eagle's medium (DMEM; no pyruvate, high glucose formulation, Gibco BRL), with 20% fetal bovine serum (FBS; Hyclone), 0.1 mM β-mercaptoethanol (Sigma), 1% non-essential amino acid stock (Gibco BRL). Generally, primate ES cells are isolated on a confluent layer of murine embryonic fibroblast in the presence of ES cell medium. In one example, embryonic fibroblasts are obtained from 12 day old fetuses from outbred mice (such as CF1, available from SASCO), but other strains may be used as an alternative. Tissue culture dishes treated with 0.1% gelatin (type I; Sigma) can be utilized. Distinguishing features of ES cells, as compared to the committed “multipotential” stem cells present in adults, include the capacity of ES cells to maintain an undifferentiated state indefinitely in culture, and the potential that ES cells have to develop into every different cell types. Unlike mouse ES cells, human ES (hES) cells do not express the stage-specific embryonic antigen SSEA-1, but express SSEA-4, which is another glycolipid cell surface antigen recognized by a specific monoclonal antibody (see, e.g., Amit et al., Devel. Biol. 227:271-278, 2000).

For rhesus monkey embryos, adult female rhesus monkeys (greater than four years old) demonstrating normal ovarian cycles are observed daily for evidence of menstrual bleeding (day 1 of cycle=the day of onset of menses). Blood samples are drawn daily during the follicular phase starting from day 8 of the menstrual cycle, and serum concentrations of luteinizing hormone are determined by radioimmunoassay. The female is paired with a male rhesus monkey of proven fertility from day 9 of the menstrual cycle until 48 hours after the luteinizing hormone surge; ovulation is taken as the day following the luteinizing hormone surge. Expanded blastocysts are collected by non-surgical uterine flushing at six days after ovulation. This procedure generally results in the recovery of an average 0.4 to 0.6 viable embryos per rhesus monkey per month (Seshagiri et al., Am J Primatol. 29:81-91, 1993).

For marmoset embryos, adult female marmosets (greater than two years of age) demonstrating regular ovarian cycles are maintained in family groups, with a fertile male and up to five progeny. Ovarian cycles are controlled by intramuscular injection of 0.75 g of the prostaglandin PGF2a analog cloprostenol (Estrumate, Mobay Corp, Shawnee, Kans.) during the middle to late luteal phase. Blood samples are drawn on day 0 (immediately before cloprostenol injection), and on days 3, 7, 9, 11, and 13. Plasma progesterone concentrations are determined by ELISA. The day of ovulation is taken as the day preceding a plasma progesterone concentration of 10 ng/ml or more. At eight days after ovulation, expanded blastocysts are recovered by a non-surgical uterine flush procedure (Thomson et al., J Med Primatol. 23:333-336, 1994). This procedure results in the average production of 1.0 viable embryos per marmoset per month.

The zona pellucida is removed from blastocysts, such as by brief exposure to pronase (Sigma). For immunosurgery, blastocysts are exposed to a 1:50 dilution of rabbit anti-marmoset spleen cell antiserum (for marmoset blastocysts) or a 1:50 dilution of rabbit anti-rhesus monkey (for rhesus monkey blastocysts) in DMEM for 30 minutes, then washed for 5 minutes three times in DMEM, then exposed to a 1:5 dilution of Guinea pig complement (Gibco) for 3 minutes. After two further washes in DMEM, lysed trophectoderm cells are removed from the intact inner cell mass (ICM) by gentle pipetting, and the ICM plated on mouse inactivated (3000 rads gamma irradiation) embryonic fibroblasts.

After 7-21 days, ICM-derived masses are removed from endoderm outgrowths with a micropipette with direct observation under a stereo microscope, exposed to 0.05% Trypsin-EDTA (Gibco) supplemented with 1% chicken serum for 3-5 minutes and gently dissociated by gentle pipetting through a flame polished micropipette.

Dissociated cells are re-plated on embryonic feeder layers in fresh ES medium, and observed for colony formation. Colonies demonstrating ES-like morphology are individually selected, and split again as described above. The ES-like morphology is defined as compact colonies having a high nucleus to cytoplasm ratio and prominent nucleoli. Resulting ES cells are then routinely split by brief trypsinization or exposure to Dulbecco's Phosphate Buffered Saline (PBS, without calcium or magnesium and with 2 mM EDTA) every 1-2 weeks as the cultures become dense. Early passage cells are also frozen and stored in liquid nitrogen.

Cell lines may be karyotyped with a standard G-banding technique (such as by the Cytogenetics Laboratory of the University of Wisconsin State Hygiene Laboratory, which provides routine karyotyping services) and compared to published karyotypes for the primate species.

Isolation of ES cell lines from other primate species would follow a similar procedure, except that the rate of development to blastocyst can vary by a few days between species, and the rate of development of the cultured ICMs will vary between species. For example, six days after ovulation, rhesus monkey embryos are at the expanded blastocyst stage, whereas marmoset embryos do not reach the same stage until 7-8 days after ovulation. The rhesus ES cell lines can be obtained by splitting the ICM-derived cells for the first time at 7-16 days after immunosurgery; whereas the marmoset ES cells were derived with the initial split at 7-10 days after immunosurgery. Because other primates also vary in their developmental rate, the timing of embryo collection, and the timing of the initial ICM split, varies between primate species, but the same techniques and culture conditions will allow ES cell isolation (see U.S. Pat. No. 6,200,806, which is incorporated herein by reference for a complete discussion of primate ES cells and their production).

Human ES cell lines exist and can be used in the methods disclosed herein. Human ES cells can also be derived from preimplantation embryos from in vitro fertilized (IVF) embryos. Experiments on unused human IVF-produced embryos are allowed in many countries, such as Singapore and the United Kingdom, if the embryos are less than 14 days old. Only high quality embryos are suitable for ES isolation. Present defined culture conditions for culturing the one cell human embryo to the expanded blastocyst have been described (see Bongso et al., Hum Reprod. 4:706-713, 1989). Co-culturing of human embryos with human oviductal cells results in the production of high blastocyst quality. IVF-derived expanded human blastocysts grown in cellular co-culture, or in improved defined medium, allows isolation of human ES cells with the same procedures described above for non-human primates (see U.S. Pat. No. 6,200,806).

Somatic precursor cells can also be utilized with the methods disclosed herein. The somatic precursor cells can be isolated from a variety of sources using methods known to one skilled in the art. The somatic precursor cells can be of ectodermal, mesodermal or endodermal origin. Any somatic precursor cells which can be obtained and maintained in vitro can potentially be used in accordance with the present methods. Such cells include cells of epithelial tissues such as the skin and the lining of the gut, embryonic heart muscle cells, and neural precursor cells (Stemple and Anderson, 1992, Cell 71:973-985).

In one example, the somatic precursor cells are mesenchymal progenitor cells. Mesenchymal progenitors give rise to a very large number of distinct tissues (Caplan, J. Orth. Res 641-650, 1991). Mesenchymal cells capable of differentiating into bone and cartilage have also been isolated from marrow (Caplan, J. Orth. Res. 641-650, 1991). U.S. Pat. No. 5,226,914 describes an exemplary method for isolating mesenchymal stem cells from bone marrow.

In other examples, the somatic precursor cells are epithelial progenitor cells or keratinocytes can be obtained from tissues such as the skin and the lining of the gut by known procedures (Rheinwald, Meth. Cell Bio. 21A:229, 1980). In stratified epithelial tissue such as the skin, renewal occurs by mitosis of precursor cells within the germinal layer, the layer closest to the basal lamina. Precursor cells within the lining of the gut provide for a rapid renewal rate of this tissue. The cells can also be liver stem cells (see PCT Publication No. WO 94/08598) or kidney stem cells (see Karp et al., Dev. Biol. 91:5286-5290, 1994). The cells can also be inner ear stem cells (see Li et al., TRENDS Mol. Med. 10: 309, 2004).

In one non-limited example, neuronal precursor cells and/or glial precursor cells are utilized. Undifferentiated neural stem cells differentiate into neuroblasts and glioblasts which give rise to neurons and glial cells. During development, cells that are derived from the neural tube give rise to neurons and glia of the central nervous system (CNS). Certain factors present during development, such as nerve growth factor (NGF), promote the growth of neural cells. Methods of isolating and culturing neural stem cells and neuronal/glial progenitor cells are well known to those of skill in the art (Hazel and Muller, 1997; U.S. Pat. No. 5,750,376). Methods for isolating and culturing neuronal precursor cells are disclosed, for example, in U.S. Pat. No. 6,610,540.

The methods disclosed herein can be used to increase the survival and expansion of any somatic precursor cells of interest. For example, the method can also be used to increase the survival and/or proliferation of pancreatic precursor cells. Pancreatic precursor cells can be induced to differentiate into differentiated cells of the pancreas, such as pancreatic endocrine cells. The method can also be used to increase the survival and/or proliferation of hepatic precursor cells.

The method includes contacting the cells with a p38 inhibitor and/or a JAK inhibitor. In one example, the cells are contacted with a p38 inhibitor. In one example, contacting the cells with a p38 inhibitor and/or a JAK inhibitor results in an increase in the phosphorylation of serine727 of STAT3 as compared to a control. In another embodiment, contacting the cells with the p38 inhibitor and/or the JOAK inhibitor results in a decrease in the phosphorylation of tyrosine705 of STAT3 as compared to a control. Suitable controls include a cell not contacted with the agent of interest, cells contacted with a carrier, cells contacted with an agent known not to affect phosphorylation of serine727 and/or tyrosine705 of STAT3, or a standard value.

Inhibitors of p38 are well known in the art. For example, PCT publication WO 95/31451 describes pyrazole compounds that inhibit MAPKs, including p38. Other p38 inhibitors have been produced, including those described in PCT Publication No. PCT Publication No. WO 98/27098, PCT Publication No. WO 99/00357, PCT Publication No. WO 99/10291, PCT Publication No. WO 99/58502, PCT Publication No. WO 99/64400, PCT Publication No. WO 00/17175, PCT Publication No. WO 00/17204, U.S. Pat. No. 6,809,199 and U.S. Pat. No. 6,759,535. An exemplary inhibitor is 4-(4-Fluorophenyl)-2-(4-methylsulfinylphenyl)-5-(4-pyridyl)1H-imidazole.

It is disclosed herein that p38 regulates cell survival. Without being bound by theory, p38 is a mediator of bone morphogenic protein (BMP) activity. BMP in turn acts through SMADs, which mediate the cellular response to transforming growth factor beta. Thus, a p38 inhibitor can be used to antagonize the effect of endogenous BMPs (which promote differentiation) or SMADs in cells, such as in stem cells or precursor cells. It is believed that p38 is downstream of noggin and gremlin in an intracellular pathway. Thus, inhibition of p38 is a more effective way of blocking any pro-differentiation effects of BMPs on stem cells or precursor cells.

In another example, the cells are contacted with a Janus kinase (JAK) inhibitor. Inhibitors of JAK are well known in the art, see for example, U.S. Pat. No. 6,452,005. In addition, bis monocyclic, bicyclic or heterocyclic aryl compounds (PCT Publication No. WO 92/20642), vinylene-azaindole derivatives (PCT Publication No. WO 94/14808) and 1-cyclopropyl-4-pyridyl-quinolones (U.S. Pat. No. 5,330,992) have been described the use of these agents as tyrosine kinase inhibitors. Styryl compounds (U.S. Pat. No. 5,217,999), styryl-substituted pyridyl compounds (U.S. Pat. No. 5,302,606), certain quinazoline derivatives (published EP Application No. 0 566 266 A1), seleoindoles and selenides (PCT Publication No. WO 94/03427), tricyclic polyhydroxylic compounds (PCT Publication No. WO 92/21660) have also been disclosed to be tyrosine kinase inhibitors. An exemplary JAK inhibitor is 2-(1,1-Dimethylethyl)-9-fluoro-3,6-dihydro-7H-benz[h]-imidaz[4,5-f]isoquinolin-7-one. A JAK inhibitor and a p38 inhibitor can be used in combination in the methods disclosed herein.

Stem cells and/or somatic precursor cells can be contacted with a JAK inhibitor and/or a p38 inhibitor in vivo or in vitro. The stem cells or somatic precursor cells can be contacted with the JAK inhibitor and/or the p38 inhibitor alone or the inhibitors alone or in conjunction with another agent. Suitable agents include cytokines and growth factors. Exemplary growth factors of use include insulin, epidermal growth factor (EGF), platelet derived growth factor (PDGF), insulin-like growth hormone (IGF)-1, growth hormone, or a fibroblast growth factor, such as FGF-2, FGF-4 and FGF-8. Exemplary agents of use also include neurotropic factors such as nerve growth factor and brain derived neurotrophic factor (BDNF), vascular endothelial growth factor (VEGF) CNTF antagonists, BMP antagonists, Notch ligands or Notch agonists (see U.S. Pat. No. 5,780,300.

Agonists of the Notch pathway are able to activate the Notch pathway at the level of protein-protein interaction or protein-DNA interaction. Agonists of Notch include but are not limited to proteins including portions of toporythmic proteins such as F3/Contactin or Delta or Serrate or Jagged (Lindsell et al., Cell 80:909-917, 1995) that mediate binding to Notch, and nucleic acids encoding the foregoing (which can be administered to express their encoded products in vivo). Thus, agonists of the Notch pathway include, but are not limited to, Notch ligands.

Jagged (also called Serrate-1) is also a Notch ligand (see Artavanis-Tsakonas et al., Annual Review of Cell Biology 7: 427-452, 1991; U.S. Pat. No. 6,083,904, U.S. Pat. No. 6,149,902, and U.S. Pat. No. 5,780,3000, which are herein incorporated by reference). Delta is another Notch ligand. Delta proteins and nucleic acids are disclosed in U.S. Pat. No. 6,783,956, which is incorporated herein by reference.

Exemplary Notch Ligands are shown in Table 1:

| Notch Ligand | GENBANK ® Accession No.¹ |
|---|---|
| Human Delta-1 | NM_005618 (Aug. 20, 2006) |
| Human Delta-3 | NM_016941 (Aug. 13, 2006) |
| Human Delta-4 | NM_019074 (Aug. 20, 2006) |
| Human Jagged-1 | NM_000214 (Aug. 20, 2006) |
| Human Jagged-2 | NM_002226 (Aug. 20, 2006) |
| Human DNER | NM_139072 (Aug. 20, 2006) |
| Human F3/contactin | NM_001843 (Aug. 20, 2006) |
| Mouse Delta-1 | NM_007865 (Aug. 27, 2006) |
| Mouse Delta-3 | NM_007866 (Jul. 16, 2006) |
| Mouse Delta-4 | NM_019454 (Jul. 16, 2006) |
| Mouse Jagged-1 | NM_013822 (Aug. 20, 2006) |
| Mouse Jagged-2 | NM_010588 (Jul. 16, 2006) |
| Mouse DNER | NM_152915 (May 14, 2006) |
| Mouse F3/contactin | NM_007727 (Feb. 12, 2006) |

¹All GENBANK ® data is incorporated by refernece herein. Dates expressed as month-day-year.

Conservative variants of these amino acid sequences, such as at most 1, at most 2, at most 3, at most 5 or at most ten conservative amino acid substitutions in the amino acid sequences set forth in the above list are also included, wherein the variants bind Notch and induce cell signalling through Notch, can also be used in the methods disclosed herein.

In one embodiment, the Notch agonist is a functionally active fragment of a protein, such as a fragment of a Notch ligand that mediates binding to Notch. In another embodiment, the agonist is a full-length protein or portion thereof (such as human Delta). In an additional embodiment, the Notch antagonist is a chimeric protein including a functional fragment of a Notch ligand and a heterologous polypeptide. Nucleic acids encoding these Notch agonists are also of use.

In one example, the Notch agonist is a fusion protein including the extra-cellular domain of Delta and an immunoglobulin constant domain. In yet another embodiment the agonist is Deltex or Suppressor of Hairless. In another embodiment, a recombinant Notch agonist is a chimeric Notch protein which comprises the intracellular domain of Notch and the extracellular domain of another ligand-binding surface receptor. For example, a chimeric Notch protein comprising the EGF receptor extracellular domain and the Notch intracellular domain has been described. Exemplary agonists and ligands are described in detail in U.S. Pat. No. 5,780,300, U.S. Pat. No. 6,703,221, and Murata-Oh et al., Int. J. Molec. Med. 13: 419-423, 2004, which are incorporated by reference in their entirety. The Notch ligand, fragment thereof, or chimeric Notch protein can include a human or a mouse Notch ligand or fragment thereof. In a further example, a nucleic acid encoding Deltex or Suppressor of Hairless is utilized in the method disclosed herein. It should be noted that any of the Notch ligands described above are of use in any of the methods disclosed herein.

A method is disclosed herein for increasing the number of neuronal stem cells or progenitor cells. The method includes contacting neuronal precursor cells with a therapeutically effective amount of (1) a Notch ligand and (2) a JAK inhibitor, a p38 inhibitor, or both, thereby increasing the survival and proliferation of the neuronal precursor cells or stem cells. The cells can be any mammalian cells of interest, including but not limited to primate cells such as human cells.

In one embodiment, stem cells and/or precursor cells are contacted with a JAK inhibitor and/or a p38 inhibitor in vitro. Generally, the JAK inhibitor or p38 inhibitor is included in a physiologically acceptable carrier, such as a tissue culture media or balanced salt solution and introduced into the cultured cells.

In another embodiment, stem cells and/or somatic precursor cells and contacted with a JAK inhibitor and/or a p38 inhibitor in vivo. Suitable subjects include those subjects that would benefit from proliferation of stem cells or precursor cells. In one embodiment, the subject is in need of proliferation of neuronal precursor cells and/or glial precursor cells. For example, the subject can have a neurodegenerative disorder or have had an ischemic event, such as a stroke. Specific, non-limiting examples of a neurodegenerative disorder are Alzheimer's disease, Pantothenate kinase associated neurodegeneration, Parkinson's disease, Huntington's disease (Dexter et al., Brain 114:1953-1975, 1991), HIV encephalopathy (Miszkziel et al., Magnetic Res. Imag. 15:1113-1119, 1997), and amyotrophic lateral sclerosis. Suitable subject also include those subjects that are aged, such as individuals who are at least about 65, at least about 70, at least about 75, at least about 80 or at least about 85 years of age. In additional examples, the subject can have a spinal cord injury, Batten's disease or spina bifida. In further examples, the subject can have hearing loss, such as a subject who is deaf, or can be in need of the proliferation of proliferation of stem cells from the inner ear to prevent hearing loss.

The methods can also be used in association with procedures such as a surgical nerve graft, or other implantation of neurological tissue, to promote healing of the graft or implant, and promote incorporation of the graft or implant into adjacent tissue. According to another aspect, the compositions could be coated or otherwise incorporated into a device or biomechanical structure designed to promote nerve regeneration. In additional embodiments, spinal cord precursor cells are treated with a therapeutically effective amount of p38 and/or a therapeutically effective amount of JAK inhibitor in vitro. A therapeutically effective amount of the cells is then transplanted into a subject of interest, such as a subject with a spinal cord injury or spina bifida. In further embodiments,

The administration can be systemic or local. In one specific, non-limiting example, the p38 inhibitor and/or JAK inhibitor is administered by injection into a ventricle of the central nervous system and/or into the spinal cord. However, any local administration can be of use, such as administration to the pancreas, into the hepatic vein or administration into the cerebral spinal fluid.

Compositions including a therapeutic moiety, such as, but not limited to, a p38 inhibitor and/or a JAK inhibitor, can be delivered by way of a pump (see Langer, supra; Sefton, CRC Crit. Ref. Biomed. Eng. 14:201, 1987; Buchwald et al., Surgery 88:507, 1980; Saudek et al., N. Engl. J. Med. 321:574, 1989) or by continuous subcutaneous infusions, for example, using a mini-pump. An intravenous bag solution can also be employed. One factor in selecting an appropriate dose is the result obtained, as measured by the methods disclosed here, as are deemed appropriate by the practitioner. Other controlled release systems are discussed in Langer (Science 249:1527-33, 1990).

In one example, a pump is implanted (for example see U.S. Pat. Nos. 6,436,091; 5,939,380; and 5,993,414). Implantable drug infusion devices are used to provide patients with a constant and long-term dosage or infusion of a therapeutic agent. Such device can be categorized as either active or passive.

Active drug or programmable infusion devices feature a pump or a metering system to deliver the agent into the patient's system. An example of such an active infusion device currently available is the Medtronic SYNCHROMED™ programmable pump. Passive infusion devices, in contrast, do not feature a pump, but rather rely upon a pressurized drug reservoir to deliver the agent of interest. An example of such a device includes the Medtronic ISOMED™.

In particular examples, compositions including a disclosed therapeutic agent are administered by sustained-release systems. Suitable examples of sustained-release systems include suitable polymeric materials (such as, semi-permeable polymer matrices in the form of shaped articles, for example films, or microcapsules), suitable hydrophobic materials (for example as an emulsion in an acceptable oil) or ion exchange resins, and sparingly soluble derivatives (such as, for example, a sparingly soluble salt). Sustained-release compositions can be administered orally, parenterally, intracistemally, intraperitoneally, topically (as by powders, ointments, gels, drops or transdermal patch), or as an oral or nasal spray. Sustained-release matrices include polylactides (U.S. Pat. No. 3,773,919, EP 58,481), copolymers of L-glutamic acid and gamma-ethyl-L-glutamate (Sidman et al., Biopolymers 22:547-556, 1983, poly(2-hydroxyethyl methacrylate)); (Langer et al., J. Biomed. Mater. Res. 15:167-277, 1981; Langer, Chem. Tech. 12:98-105, 1982, ethylene vinyl acetate (Langer et al., Id.) or poly-D-(−)-3-hydroxybutyric acid (EP 133,988).

Polymers can be used for ion-controlled release. Various degradable and nondegradable polymeric matrices for use in controlled drug delivery are known in the art (Langer, Accounts Chem. Res. 26:537, 1993). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425, 1992; and Pec, J. Parent. Sci. Tech. 44(2):58, 1990). Alternatively, hydroxyapatite has been used as a microcarrier for controlled release of proteins (Ijntema et al., Int. J. Pharm. 112:215, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa., 1993). Numerous additional systems for controlled delivery of therapeutic proteins are known (for example, U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; and U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342; and U.S. Pat. No. 5,534,496).

In another embodiment, the subject is in need of a proliferation of pancreatic precursor cells. Suitable subject include, but are not limited to, subjects with type I or type II diabetes. In a further embodiment, the subject is in need of a proliferation of ectodermal precursor cells. Suitable subjects include those with wounds or fractures. In a further embodiment, the subject is in need of a proliferation of hepatic precursor cells. Suitable subjects are those individuals with liver disease.

As noted above, for use in any of the therapeutic methods disclosed herein, administration of the JAK inhibitor and/or p38 inhibitor (and optionally additional agents) can be systemic or local. Oral, intravenous, intra-arterial, subcutaneous, intra-peritoneal, intra-muscular, intra-ventricular, intra-nasal transmucosal, subcutaneous and even rectal administration is contemplated.

Pharmacological compositions for use can be formulated in a conventional manner using one or more pharmacologically (for example, physiologically or pharmaceutically) acceptable carriers comprising excipients, as well as optional auxiliaries that facilitate processing of the active compounds into preparations which can be used pharmaceutically. Proper formulation is dependent upon the route of administration chosen.

Thus, for injection, the active ingredient can be formulated in aqueous solutions, preferably in physiologically compatible buffers. For transmucosal administration, penetrants appropriate to the barrier to be permeated are used in the formulation. Such penetrants are generally known in the art. For oral administration, the active ingredient can be combined with carriers suitable for inclusion into tablets, pills, dragees, capsules, liquids, gels, syrups, slurries, suspensions and the like. A p38 inhibitor and/or a JAK inhibitor can also be formulated for use in inhalation therapy. For administration by inhalation, the active ingredient is conveniently delivered in the form of an aerosol spray presentation from pressurized packs or a nebuliser, with the use of a suitable propellant.

The JAK inhibitor and/or the p38 inhibitor can be formulated for parenteral administration by injection, such as by bolus injection (a pulsatile dose) or continuous infusion. Similarly, JAK inhibitor and/or the p38 inhibitor can be formulated for intratracheal or for inhalation. Such compositions can take such forms as suspensions, solutions or emulsions in oily or aqueous vehicles, and can contain formulatory agents such as suspending, stabilizing and/or dispersing agents. Other pharmacological excipients are known in the art.

Therapeutically effective doses of the presently described compounds can be determined by one of skill in the art, with a goal of achieving a desired level of proliferation of stem cells and/or somatic precursor cells. The relative toxicities of the compounds make it possible to administer in various dosage ranges. In one example, the compound is administered orally in single or divided doses.

The specific dose level and frequency of dosage for any particular subject may be varied and will depend upon a variety of factors, including the activity of the specific compound, the extent of existing disease activity, the age, body weight, general health, sex, diet, mode and time of administration, rate of excretion, drug combination, and severity of the condition of the host undergoing therapy.

### Method for Increasing the Number of Neuronal Cells In Vivo

A method is disclosed herein for increasing the proliferation/survival of neuronal precursor cells in vivo. The method includes administering to a subject a therapeutically effective amount of a Notch ligand and a growth factor. The survival and/or proliferation of central nervous system precursor cells and/or peripheral nervous system precursor cells can be induced using the methods disclosed herein.

The subject can be any subject of interest. Suitable subjects include those subjects that would benefit from proliferation of stem cells or precursor cells. In one embodiment, the subject is in need of proliferation of neuronal precursor cells and/or glial precursor cells. For example, the subject can have a neurodegenerative disorder or have had an ischemic event, such as a stroke. Specific, non-limiting examples of a neurodegenerative disorder are Alzheimer's disease, Pantothenate kinase associated neurodegeneration, Parkinson's disease, Huntington's disease (Dexter et al., Brain 114:1953-1975, 1991), HIV encephalopathy (Miszkziel et al., Magnetic Res. Imag. 15:1113-1119, 1997), and amyotrophic lateral sclerosis. Suitable subject also include those subjects that are aged, such as individuals who are at least about 65, at least about 70, at least about 75, at least about 80 or at least about 85 years of age. In additional examples, the subject can have a spinal cord injury, Batten's disease or spina bifida. In further examples, the subject can have hearing loss, such as a subject who is deaf, or can be in need of the proliferation of proliferation of stem cells from the inner ear to prevent hearing loss.

The methods can also be used in association with procedures such as a surgical nerve graft, or other implantation of neurological tissue, to promote healing of the graft or implant, and promote incorporation of the graft or implant into adjacent tissue. According to another aspect, the compositions could be coated or otherwise incorporated into a device or biomechanical structure designed to promote nerve regeneration. In additional embodiments, spinal cord precursor cells are treated with a therapeutically effective amount of a Notch ligand, and a therapeutically effective amount of a growth factor in vitro. The cells are then transplanted into a subject, such as a subject with a spinal cord injury or spina bifida.

Agonists of the Notch pathway are able to activate the Notch pathway at the level of protein-protein interaction or protein-DNA interaction. Agonists of Notch include but are not limited to proteins including portions of toporythmic proteins such as Delta or Serrate or Jagged (Lindsell et al., Cell 80:909-917, 1995) that mediate binding to Notch, and nucleic acids encoding the foregoing (which can be administered to express their encoded products in vivo). In specific non-limiting examples, the Notch ligand is F3/Contactin, Delta or Jagged. Combinations of Notch ligands can also be utilized. The amino acid sequences of Notch ligand are well known in the art, see for example GENBANK® Accession No. NM_{—}019454 (Jul. 16, 2006) and NM_{—}019147 (Apr. 23, 2006), which are incorporated herein by reference.

In one embodiment, the agonist is a functionally active fragment, such as a fragment of a Notch ligand that mediates binding to Notch. In another embodiment, the agonist is a human protein or portion thereof (such as human Delta). In yet another embodiment the agonist is Deltex or Suppressor of Hairless, or a nucleic acid encoding Delta, Jagged, Deltex or Suppressor of Hairless. In another embodiment, a recombinant Notch agonist is a chimeric Notch protein which comprises the intracellular domain of Notch and the extracellular domain of another ligand-binding surface receptor. For example, a chimeric Notch protein comprising the EGF receptor extracellular domain and the Notch intracellular domain has been described. These agonists and ligands are described in detail in U.S. Pat. No. 5,780,300, which is incorporated by reference in its entirety.

The method includes the administration of a therapeutically effective amount of a growth factor. Suitable growth factors include fibroblast growth factor, epithelial growth factor, nerve growth factor, brain derived neurotrophic factor, vascular endothelial growth factor, and cilliary neurotrophic factor, amongst others. Combinations of growth factors can also be utilized.

In one embodiment, the growth factor is a fibroblast growth factor, such as, but not limited to, FGF-2 or FGF-4. Thus, in specific non-limiting examples, a therapeutically effective amount of Delta and FGF-2, Jagged and FGF-2, Delta and FGF-4, or Jagged and FGF-4, are administered to a subject. In additional embodiments the growth factor is insulin, epidermal growth factor, platelet derived growth factor.

In one embodiment, the Notch ligand and the growth factor are administered systemically. In another embodiment, the Notch ligand and the growth factor are administered locally. Local administration includes, but is not limited to, injection into a ventricle of the brain. Formulations and systems for the delivery of therapeutic agents are described above. For example, carriers, buffers, routes of administration, sustained release systems, and pumps for the delivery of therapeutic agents are disclosed above.

In one non-limiting example, the method includes intraventricular infusion of a Notch ligand and a growth factor into the central nervous system, or into the cerebral spinal fluid. Alternatively, the method can include interstitial delivery to the central nervous system. For example, the Notch ligand and the growth factor can be introduced using a cannula and an osmotic pump. The Notch ligand and the growth factor can be infused intraventricularly using an Ommaya reservoir, a plastic reservoir implanted subcutaneously in the scalp and connected to the ventricles within the brain via an outlet catheter. Solutions can be subcutaneously injected into the implanted reservoir and delivered to the ventricles by manual compression of the reservoir through the scalp. Several implantable pumps have been developed that possess several advantages over the Ommaya reservoir. These can be implanted subcutaneously and refilled by subcutaneous injection and are capable of delivering drugs as a constant infusion over an extended period of time. Furthermore, the rate of drug delivery can be varied using external handheld computer control units.

Pharmaceutical preparations and dosing are disclosed above. The subject can be any mammalian subject of interest, including but not limited to human subjects. In several examples, the subject has a neurodegenerative disorder. In other embodiments, the subject has a traumatic injury or a stroke. In several examples, a therapeutically effective amount of a Notch ligand and a growth factor are administered to a subject, such as a subject with Parkinson's disease, Alzheimer's disease, or a stroke. The administration of the Notch ligand and the growth factor results in the amelioration of a sign or a symptom of the disorder.

### Screening

A method is provided herein for identifying an agent that alters the survival or proliferation of stem cells or precursor cells. The method includes contacting a stem cell expressing STAT3 and/or a precursor cell expressing STAT3 with an effective amount of an agent of interest. The phosphorylation status of serine 727 of STAT3 is determined. Increased phosphorylation of serine 727 of STAT3 in the stem cell and/or the precursor cells indicates that the agent increases the survival or proliferation of stem cells and/or or precursor cells. The method can also include assessing the phosphorylation of tyrosine 705 of STAT3. A decrease in the phosphorylation of tyrosine 705 in the stem cells and/or the precursor cells contacted with the agent indicates that the agent is of use to increase the survival or proliferation of stem cells and/or precursor cells. The cell can be any stem cell of interest, such as an embryonic stem cell. The cell can be any precursor cell of interest, such as a somatic precursor cell. In several examples, the somatic precursor cells is a neuronal precursor cell, a glial precursor cell, or a pancreatic precursor cell. In additional examples, the cell is any mammalian cell, such as a human cell. In additional examples, the cell expresses sonic hedgehog. In additional examples, the cells express Hes3. In further examples, the cells express nucleostemin.

Decreased phosphorylation of serine 727 of STAT3 indicates that the agent decreases the survival and/or proliferation of stem cells and/or precursor cells. The amino acid sequence of STAT3 is well known to those of skill in the art. Exemplary amino acid sequences of STAT3 can be found, for example, as GENBANK® Accession Nos. NP_{—}644805 (Unigene, see also GENBANK® No. A54444, Jul. 28, 2000), NP_{—}003141 (Sep. 3, 2006), NP_{—}998825 (Aug. 25, 2006), NP_{—}998824 (Aug. 24, 2006), AAH00627 (Jul. 15, 2006), AAH87025 (Jul. 16, 2006), CAA62920 (Apr. 8, 2005), which are all incorporated herein by reference.

The phosphorylation of serine 727 of STAT3 in the stem cell or precursor cell contacted with the agent can be compared to a control. Suitable controls include the phosphorylation status of serine 727 in a cell not contacted with the agent, or in cells contacted with a control agent, such as a vehicle, or a compound known not to affect the phosphorylation of serine 727 of STAT3. Suitable controls also include standard values. It should be noted that the assay can be performed on intact cells or cell extracts.

In another embodiment, a method is provided for identifying an agent that alters the survival and/or proliferation of neoplastic cells, such as tumor cells. The method includes contacting a neoplastic cell, such as a tumor cell expressing STAT3 with an agent of interest. The phosphorylation status of serine 727 of STAT3 is determined. Decreased phosphorylation of serine 727 of STAT3 in the neoplastic cell, such as the tumor cells, indicates that the agent indicates that the agent is of use as a chemotherapeutic agent.

Tumors include, but are not limited to, a cancer of the adrenal gland, bladder, bone, bone marrow, brain, breast, central nervous system, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. Exemplary cancers include adenocarcinoma, leukemia, lymphoma, melanoma, myeloma, sarcoma, teratocarcinoma, hyperplasia and hypertrophy. Exemplary cancers also include ACTH-producing tumors, acute lymphocytic leukemia, acute nonlymphocytic leukemia, cancer of the adrenal cortex, bladder cancer, brain cancer, breast cancer, cervical cancer, chronic lymphocytic leukemia, chronic myelocytic leukemia, colorectal cancer, cutaneous T-cell lymphoma, endometrial cancer, esophageal cancer, Ewing's sarcoma, gallbladder cancer, gliomas, hairy cell leukemia, head & neck cancer, Hodgkin's lymphoma, Kaposi's sarcoma, kidney cancer, liver cancer, lung cancer (small and/or non-small cell), malignant peritoneal effusion, malignant pleural effusion, melanoma, mesothelioma, multiple myeloma, neuroblastoma, non-Hodgkin's lymphoma, osteosarcoma, ovarian cancer, ovary (germ cell) cancer, prostate cancer, pancreatic cancer, penile cancer, retinoblastoma, skin cancer, soft-tissue sarcoma, squamous cell carcinomas, stomach cancer, testicular cancer, thyroid cancer, trophoblastic neoplasms, uterine cancer, vaginal cancer, cancer of the vulva, and Wilm's tumor. In one example, the tumor is a glioblastoma or a hemangioblastoma.

The test compound can be any compound of interest, including chemical compounds, small molecules, polypeptides, growth factors, cytokines, or other biological agents (for example antibodies). In several examples, a panel of potential chemotherapeutic agents, or a panel of potential neurotrophic agents are screened. In other embodiments a panel of polypeptide variants is screened. The test compound can be a Notch agonist or a Notch antagonist.

Methods for preparing a combinatorial library of molecules that can be tested for a desired activity are well known in the art and include, for example, methods of making a phage display library of peptides, which can be constrained peptides (see, for example, U.S. Pat. No. 5,622,699; U.S. Pat. No. 5,206,347; Scott and Smith, Science 249:386-390, 1992; Markland et al., Gene 109:13-19, 1991), a peptide library (U.S. Pat. No. 5,264,563); a peptidomimetic library (Blondelle et al., Trends Anal Chem. 14:83-92, 1995); a nucleic acid library (O'Connell et al., Proc. Natl. Acad. Sci., USA 93:5883-5887, 1996; Tuerk and Gold, Science 249:505-510, 1990; Gold et al., Ann. Rev. Biochem. 64:763-797, 1995); an oligosaccharide library (York et al., Carb. Res. 285:99-128, 1996; Liang et al., Science 274:1520-1522, 1996; Ding et al., Adv. Expt. Med. Biol. 376:261-269, 1995); a lipoprotein library (de Kruif et al., FEBS Lett. 3 99:23 2-23 6, 1996); a glycoprotein or glycolipid library (Karaoglu et al., J Cell Biol. 130.567-577, 1995); or a chemical library containing, for example, drugs or other pharmaceutical agents (Gordon et al., J. Med. Chem. 37.1385-1401, 1994; Ecker and Crooke, BioTechnology 13:351-360, 1995). Polynucleotides can be particularly useful as agents that can alter a function of stem cells (such as, but not limited to ES cells) and precursor cells (such as, but not limited to, pancreatic precursor cells and neuronal precursor cells) because nucleic acid molecules having binding specificity for cellular targets, including cellular polypeptides, exist naturally, and because synthetic molecules having such specificity can be readily prepared and identified (see, for example, U.S. Pat. No. 5,750,342).

In one embodiment, for a high throughput format, stem or precursor cells can be introduced into wells of a multiwell plate or of a glass slide or microchip, and can be contacted with the test agent. Generally, the cells are organized in an array, particularly an addressable array, such that robotics conveniently can be used for manipulating the cells and solutions and for monitoring the stem or precursor cells, particularly with respect to the function being examined. An advantage of using a high throughput format is that a number of test agents can be examined in parallel, and, if desired, control reactions also can be run under identical conditions as the test conditions. As such, the methods disclosed herein provide a means to screen one, a few, or a large number of test agents in order to identify an agent that can alter a function of cells, for example, an agent that induces the cells to differentiate into a desired cell type, or that prevents spontaneous differentiation and allows proliferation.

In assays that use cells, the cells are contacted with test compounds. In some embodiments, the cells are incubated with the test compound for an amount of time sufficient to affect phosphorylation of a substrate, such as STAT3, by a kinase. The cells are lysed and the amount of phosphorylated STAT3 is measured. The amounts of phosphorylated STAT3 that is present in the cells is compared to identical cells that were not exposed to the test compound. Specifically, the phosphorylation of STAT3 at serine 727 is measured. In some embodiments, the phosphorylation at tyrosine 705 is also measured. Thus, the phosphorylation of STAT3 at serine 727 can be compared to the phosphorylation of STAT3 at tyrosine 705, if desired.

A method is also provided herein to determine if a tumor is invasive and or proliferating. The method can be used to assess if a tumor is benign or malignant. The method can also be used in the diagnosis of tumors, or for determining the prognosis of a subject. The method can also be used to determine the effectiveness of a therapeutic regimen, such as, but not limited to, the administration of one or more chemotherapeutic agents and/or radiation. Exemplary types of tumors are listed above.

In one embodiment, the method includes obtaining a sample including tumor cells, and determining the phosphorylation status of serine 727 of STAT3 in the tumor cells. Phosphorylation of serine 727 of STAT3 in the tumor cells as compared to the phosphorylation of tyrosine 705 of STAT3 indicates that the tumor is proliferating and/or invasive. The phosphorylation of serine 727 of STAT3 can be compared to a control. Suitable controls include, but are not limited to, the phosphorylation of STAT3 in a tumor known not to be invasive or the phosphorylation of STAT3 in a tumor known to be non-invasive. Generally the phosphorylation of STAT3 is assessed from a sample including approximately the same number of cells from the same type of tumor, although this is not an absolute requirement. The control can also be a standard value. The sample can be any sample of interest that includes tumor cells. In several embodiments, the sample is a biopsy, an aspiration from a solid tumor, blood or a bone marrow sample.

In one example, increased phosphorylation of serine 727 of STAT3 as compared to the phosphorylation of tyrosine 705 of STAT3 indicates that the tumor is malignant. Malignant cancer is a subset of neoplastic disorders that show a greater degree of anaplasia and have the properties of invasion and metastasis. In another example, increased phosphorylation of serine 727 of STAT3 as compared to the phosphorylation of tyrosine 705 of STAT3 indicates that the undifferentiated stem cells are present in the tumor.

In another example, decreased phosphorylation of serine 727 of STAT3 as compared to tyrosine 705 of STAT3 indicates a low level of proliferation of the tumor cells. In one example, decreased phosphorylation of serine 727 of STAT3 as compared to tyrosine 705 of STAT3 indicates that the tumor is benign.

In specific non-limiting example, the invasiveness and/or ability to proliferate of a tumor of the central nervous system are/is assessed using the methods disclosed herein. A sample including cells from the tumor of the central nervous system is obtained, and the phosphorylation status of serine 727 of STAT3 in the tumor cells is determined. Phosphorylation of serine 727 of STAT3 in the tumor cells as compared to the phosphorylation of tyrosine 705 of STAT3 indicates that the central nervous system tumor is proliferating and/or invasive.

Optionally, the number or presence of CNS stem cells is also assessed. The number and/or presence of CNS stem cells can be assessed by any method known to one of skill in the art. In one exemplary method for assessing the presence of CNS stem cells, a sample, such as a biopsy, of the CNS tumor is obtained. The tumor is mechanically dissociated into single cells in a suitable media with appropriate growth factors, such as N2 tissue culture media including and FGF-2 (for example, at about 20 ng/ml). The cell suspension is plated in N2 medium including FGF-2 (about 20 ng/ml) and allowed to proliferate to generate a suitable number of cells. In one example, the cells are culture for about four days to about two weeks, such as for about one week. The culture is then dissociated in a basic medium without calcium or magnesium, such as Hank's Buffered Saline Solution (HBSS). The cells are then re-plated in N2 medium supplemented with FGF-2. The number of CNS stem cells produced in these culture conditions can then be enumerated.

Alzheimer's disease is characterized by a progressive loss of neurons, formation of fibrillary tangles within neurons, and the formation of plaques in affected regions of the brain. The key pathogenic event is likely the excessive formation and accumulation of amyloid peptide (αβ), which are a set of 39-43 amino acid peptides produced by the cleavage of a glycoprotein, β-amyloid precursor protein (APP) by secretases, such as γ-secretase. Amyloid peptides are toxic in vitro. It has been proposed that amyloid peptides are directly toxic. It has also been proposed that amyloid peptides damage neuron indirectly by damaging glial cells. It has been shown that αβ binds p75, which bind NGF and other neurotrophins. Thus, αβ could induce cell death through p75.

It is disclosed herein that APP can also lead to changes of the phosphorylation of STAT3. Thus, the accumulation of αβ results in decreased phosphorylation of serine 727 of STAT3. The presence of APP results in increased phosphorylation of serine 727 of STAT3, leading to increased cell survival. The presence of αβ leads to decreased phosphorylation of serine 727 of STAT3, leading to decreased cell survival. Without being bound by theory, a therapeutically effective amount p38 inhibitor and/or JAK inhibitor can be administered to a subject with Alzheimer's disease to increase the phosphorylation of serine 727 of STAT3 and to increase neuronal cell survival.

Thus, a method is provided herein for identifying an agent that can be used to treat Alzheimer's disease. The method includes obtaining a sample of cells of the central nervous system and contacting the cell with an effective amount of agent of interest. The phosphorylation status of serine 727 of STAT3 is assessed in the cells. Phosphorylation of serine 727 of STAT3 indicates that the agent is of use in treating Alzheimer's disease. The phosphorylation of serine 727 of STAT3 can be compared to a control, such as, but not limited to, a sample not contacted with the agent of interest, a sample contacted with vehicle, or a standard value. Any agent of interest can be tested using the methods disclosed herein, including chemical compounds, small molecules, polypeptides, growth factors, cytokines, or other biological agents (for example antibodies). In several examples, a panel of potential chemotherapeutic agents, or a panel of potential neurotrophic agents are screened. In other embodiments a panel of polypeptide variants is screened. The test compound can be a Notch ligand and/or a modulator of the p75 receptor. The p75 receptor is a member of the tumor necrosis factor superfamily; p75 specifically binds neurotrophins. All of the neurotrophins bind to p75 with a similar nanomolar affinity, but with different kinetics.

In one embodiment, Notch ligands are screened to determine if they alter the phosphorylation of serine 727 of STAT3. In another embodiment, cytokines and/or growth factors are screened to determine if they affect the amount of APP in a cell. In addition, synergies between Notch ligands and modulators of the p75 receptor can be assessed using the methods disclosed herein.

In one example, a combination of agents is assessed using this method. In one example, a cell of the central nervous system is contacted with a Notch ligand and a growth factor, such as, but not limited to, brain derived neurotrophic factor (BDNF) or nerve growth factor (NGF). In another example, a cell of the central nervous system is contacted with a Notch ligand and a modulation of the p75 receptor. Phosphorylation of serine 727 of STAT3 is then assessed in the cells of the central nervous system. An increased phosphorylation of STAT3 as compared to a control indicates that the one or more agents increases cell survival and/or is of use in treating Alzheimer's disease.

In some embodiments, Western blot technology is used with the cell proteins separated by electrophoresis and antibodies that bind to STAT3, STAT3 phosphorylated at serine 727, and/or antibodies that specifically bind the STAT3 phosphorylated at tyrosine 705 are utilized. Alternatively, the cells may be incubated in the presence of orthophosphate containing a radiolabeled phosphorus, permitting the detection of phosphorylated or unphosphorylated substrate (such as phosphorylated STAT3, or STAT3 phosphorylated specifically at serine 727).

In some embodiments, cells are treated in vitro with test compounds at 37° C. in a 5% CO₂ humidified atmosphere. Following treatment with test compounds, cells are washed with Ca²+ and Mg²+ free PBS and total protein is extracted as described (Haldar et al., Cell Death Diff. 1:109-115, 1994; Haldar et al., Nature 342:195-198, 1989; Haldar et al., Cancer Res. 54:2095-2097, 1994). In additional embodiments, serial dilutions of test compound are used.

In some embodiments, phosphorylation is analyzed using Western blotting and immunodetection which are performed using Amersham ECL an enhanced chemiluminescence detection system and well known methodology. In one example, phosphorylation of stem cells or precursor cells can be carried out in phosphate free media (GIBCO) using 1 mCi/ml [P³²] orthophosphoric acid (NEN) for six hours in the presence of a test compound. Immunoprecipitation of P³² labeled cellular extract can be performed, for example, as described in Haldar et al., Nature 342:195-198, 1998.

Generally, immunoprecipitation utilizes an antibody that binds a substrate of interest, such as STAT3 phosphorylated at serine 727 or STAT3 phosphorylated at serine 705. An immunocomplex is run on a 0.75 mm thick 10% SDS-PAGE. Subsequently, gels are dried and exposed for autoradiography.

Phospho-amino acid analysis can be performed as is known in the art. For example, the analysis can be performed essentially as described in the manual for the Hunter thin layer electrophoresis system, HTLE700, (CBS Scientific Company Inc., USA). Briefly, P³² labeled immunoprecipitates are run on 10% SDS-PAGE gels. The immunoreactive bands of interest are cut out of the gel and eluted with 50 μM ammonium bicarbonate. After elution, the proteins are precipitated in the presence of 15%-20% TCA plus carrier protein, and washed with ethanol. Precipitated protein is then oxidized in performic acid and lyophilized. The dried pellet is resuspended in constant boiling HCl, heated at 110° C. and lyophilized. The residue is resuspended in pH 1.9 buffer (50 μl formic acid, 156 μl acetic acid, 1794 mcl H₂O) containing phospho-amino acid standards and spotted on a PEI cellulose plate. Two-dimensional thin layer chromatography is run using the pH 1.9 buffer for the first dimension and pH 3.5 buffer (100 ml acetic acid, 10 ml pyridine, 1890 ml H₂O) for the second. The plate is baked at 65° C. for 10 minutes, and the cold standards are visualized by spraying the plate with 0.25% ninhydrin and returning the plate to the 65° C. oven for 15 minutes. The plate is then exposed to film, such as to Kodak X-omat AR film, for two to four weeks.

In some embodiments, modulation of phosphorylation is analyzed using cell extract material as a starting material. Test compounds are combined with cell extract material, such as an extract from stem cells or precursor cells, and the effect of the compounds on phosphorylation of STAT3 is examined. In one example, the cell extract material is contacted with test compounds to identify the effect the test compound has on phosphorylation serine 727 in the presence of ³²P-gamma-ATP, and/or to identify the effect the test compound has on phosphorylation of tyrosine 705.

In an exemplary protocol, cell extract is treated in vitro at 37° C. using 100 μg total cellular extract with a specified concentration of test compounds. For phosphatase reactions, 50 μl cell lysate is contacted with test compound and incubated with a reaction mixture for 30-60 minutes at 37° C.

For phosphorylation of cell extract material, in one example, 100 μg cellular extract is treated as described above except 40 μci [³²P]ATP (3000 Ci/mmol) are added to each reaction. Reactions are stopped by immersing the tubes in ice. The [³²P] ATP labeled reaction mixture is absorbed on immunoaffinity column made from the monoclonal antibody against STAT3 phosphorylated at serine 727 by covalently binding purified antibodies to protein-A Sepharose using the crosslinker dimethylpimelimidate dihydrochloride (50 mM). Specifically bound [³²P]-labeled protein is eluted with 0.05 M diethylamine, pH 11.5 containing 0.5% Na-deoxycholate.

In exemplary methods, immunodetection by Western blotting is performed using Amersham ECL detection system and methodology known to one of skill in the art. Immunoprecipitation of P³² labeled cellular extract can be performed, for example, as described in Haldar et al., Nature 342:195-198, 1989. The immunocomplex is run on a 0.75 mm thick 10% SDS-PAGE. Subsequently, gels are dried and exposed for autoradiography using film such as Kodak XAR film.

Phosphoaminoacid analysis can be performed essentially as described in the manual for the Hunter thin layer electrophoresis system, HTLE700, (CBS Scientific Company Inc., USA). In an exemplary method, P³² labeled immunoprecipitates are run on 10% SDS-PAGE gels. The STAT3 immunoreactive bands are cut out of the gel and eluted with 50 μM ammonium bicarbonate. After elution the proteins are precipitated in the presence of 15%-20% TCA plus carrier protein, and washed with ethanol. Precipitated protein are then oxidized in performic acid and lyophilized. The dried pellet is resuspended in constant boiling HCl, heated at 110° C. and lyophilized. The residue is resuspended in pH 1.9 buffer (50 μl formic acid, 156 μl acetic acid, 1794 μl H₂O) containing phospho-amino acid standards and spotted on a PEI cellulose plate. Two dimensional thin layer chromatography is run using the pH 1.9 buffer for the first dimension and pH 3.5 buffer (100 ml acetic acid, 10 ml pyridine, 1890 ml H₂O) for the second. The plate is baked at 65° C. for 10 minutes, and the cold standards are visualized by spraying the plate with 0.25% ninhydrin and returning the plate to the 65° C. oven for 15 minutes. The plates are then exposed to film.

### Isolated Cell Populations

Isolated cells expressing STAT3 phosphorylated at serine 727 (STAT3ser727⁺) and nestin are disclosed herein. In several embodiments, the cells can also express Notch (for example, Notch1), Hes3, and/or nucleostemin. In additional embodiments, an isolated population of cells is provided, wherein STAT3 phosphorylated at serine 727 is detectable in the cells, and wherein STAT3 phosphorylated at tyrosine 705 is not detectable in the cells. In some examples, one or more of Notch (for example, Notch1), Hes3, and/or nucleostemin can be detected in at least 80%, at least 85%, at least 90%, at least 95%, at least 96%, at least 97%, at least 98%, at least 99% or in 100% of the cells in the isolated population.

In additional embodiments, the isolated cells express CD133 (also known as AC133). Thus, in some examples, CD133 (AC133) can be detected in at least 80%, at least 85%, at least 90%, at least 95%, at least 96%, at least 97%, at least 98%, at least 99% or in 100% of the cells in the isolated population.

In further embodiments, the isolated cells express sonic hedgehog (SHH). Thus, in some examples, SHH can be detected in at least 80%, at least 85%, at least 90%, at least 95%, at least 96%, at least 97%, at least 98%, at least 99% or in 100% of the cells in the isolated population

These isolated cells are precursor cells or stem cells, and thus can differentiate into more than one cells type. In one example, the isolated cells are stem cells. In another example, the isolated cells are precursor cells, such as neuronal or pancreatic precursor cells. In several embodiments, the composition comprises fewer than about 20%, about 10%, about 5%, or about 1% of fully differentiated cells. Thus, the composition comprises more than about 80%, about 90%, about 95% or about 99% precursor cells and/or stem cells.

Isolated cells wherein phosphorylation at serine 727 (STAT3ser727⁺) of STAT3 is detectable in the isolated cells, and wherein phosphorylation at tyrosine 705 is not detectable in the isolated cells. In additional embodiments, at least one additional marker indicated with a “+” in Table 2 is detected in the cells. In one example, expression of nestin can also be detected in the isolated cells.

TABLE 2
Additional Markers

| Antigen | Immunoreactivity |
|---|---|
| Akt (also known as protein kinase B) | + |
| 2′,3′-Cyclic Nucleotide 3′-Phosphodiesterase | − |
| (CNPase) | |
| Ciliary neurotrophic factor (CNTF) Receptor | + |
| Doublecortin | − |
| Glial fibrillar acidic protein (GFAP) | − |
| gp130 | + |
| Hes3 | + |
| Insulin-like growth factor (IGF1)/Insulin Receptor | + |
| JAK2 kinase | + |
| Lyman-Kutcher-Burman (LKB)1 | + |
| myeloid cell leukemia sequence (Mcl)-1 | + |
| mitogen- and stress-activated protein kinase | + |
| (MSK)-1 | |
| Mammalian target of rapamycin (mTOR) | + |
| Nestin | + |
| Notch1 | + |
| Notch3 | + |
| p38 microtubule activated protein kinase (MAP | + |
| kinase) | |
| Platelet derived growth factor (PDGFR)a | + |
| PDGFRb | + |
| Phosphoinositol (PI)3 kinase | + |
| Smooth muscle actin (SMA) | − |
| Sonic Hedgehog | + |
| SRY-related HMG box (Sox2) | + |
| STAT1 | + |
| STAT2 | + |
| STAT3 | + |
| TUJ1 | − |
| Vascular endothelial growth factor receptor | + |
| (VEGFR)1 | |

In several embodiments, STAT3 phosphorylated at serine 727 can be detected in the greater than about 80%, greater than about 85%, greater than about 90%, greater than about 95%, or greater than about 99% of the cells in the isolated population, and/or STAT3 phosphorylated at tyrosine 705 can be detected in less than about 80%, less than about 85%, less than about 90%, less than about 95%, or less than about 99% of the cells in the isolated population. In addition, one or more of Akt, CNTF receptor, gp130, IGF-I/insulin receptor, JAK2 kinase, LKB1, Mc1-1, MSK-1, mTOR, Notch3, p38 MAP kinase, PDGFRa, PDGFRb, PI3 kinase, sonic hedgehog, Sox2, STAT1, STAT2, STAT3, VEGF1 can be detected in the cells

In one example, expression of (1) STAT3 phosphorylated at serine 727 (STAT3ser727⁺) and nestin, (2) Notch, Hes3, and/or nucleostemin, and (3) one or more of Akt, CNTF receptor, gp130, IGF-I/insulin receptor, JAK2 kinase, LKB1, Mc1-1, MSK-1, mTOR, Notch3, p38 MAP kinase, PDGFRa, PDGFRb, PI3 kinase, sonic hedgehog, Sox2, STAT1, STAT2, STAT3, VEGF1 can be detected in greater than about 85%, greater than about 90%, greater than about 95%, or greater than about 99% of the cells in the isolated population. In another example, expression of (1) STAT3 phosphorylated at serine 727 (STAT3ser727⁺) and nestin, (2) Notch, Hes3, and/or nucleostemin, and (3) two, three, four, five, six, seven, eight, nine, ten, eleven, twelve, thirteen, fourteen, fifteen, sixteen, seventeen, eighteen, nineteen, twenty or more of Akt, CNTF receptor, gp130, IGF-I/insulin receptor, JAK2 kinase, LKB1, Mc1-1, MSK-1, mTOR, Notch3, p38 MAP kinase, PDGFRa, PDGFRb, PI3 kinase, sonic hedgehog, Sox2, STAT1, STAT2, STAT3, VEGF1 can be detected in greater than about 85%, greater than about 90%, greater than about 95%, or greater than about 99% of the cells in the isolated population. In yet another example, expression of (1) STAT3 phosphorylated at serine 727 (STAT3ser727⁺) and nestin, (2) Notch, Hes3, and/or nucleostemin, and (3) all of Akt, CNTF receptor, gp130, IGF-I/insulin receptor, JAK2 kinase, LKB1, Mc1-1, MSK-1, mTOR, Notch3, p38 MAP kinase, PDGFRa, PDGFRb, PI3 kinase, sonic hedgehog, Sox2, STAT1, STAT2, STAT3, VEGF1 can be detected in greater than about 85%, greater than about 90%, greater than about 95%, or greater than about 99% of the cells in the isolated population.

In additional examples, expression of one or more of GFAP, CNPase, smooth muscle action, and TUJ1 cannot be detected in the cells. In further examples, none of GFAP, CNPase, smooth muscle action, and TUJ1 can be detected in the cells. Thus, in some examples, STAT3 phosphorylated at serine 727 (STAT3ser727⁺) and nestin can be detected in greater than about 85%, greater than about 90%, greater than about 95%, or greater than about 99% of the cells in the isolated population, STAT3 phosphorylated at tyrosine 705 cannot be detected in greater than about 85%, greater than about 90%, greater than about 95%, or greater than about 99% of the cells in the isolated population, and one or more of GFAP, CNPase, smooth muscle actin, and TUJ1 cannot be detected in greater than about 85%, greater than about 90%, greater than about 95%, or greater than about 99% of the cells in the isolated population. In additional embodiments, one or more of Notch, Hes3 and nucleostemin is expressed in greater than about 85%, greater than about 90%, greater than about 95%, or greater than about 99% of the cells in the isolated population. In further embodiments, greater than about 80%, greater than about 90%, greater than about 95%, or greater than about 99% of cells expressing STAT3 phosphorylated at serine 727 (STAT3ser727⁺) and nestin express at least one additional marker indicated with a “+” in Table 2 in the isolated population. In other embodiments, greater than about 80%, greater than about 90%, greater than about 95%, or greater than about 99% of cells expressing STAT3 phosphorylated at serine 727 (STAT3ser727⁺) and nestin, one or more of Notch, Hes3, and nucleostemin, one additional marker indicated with a “+” in Table 2, and AC133.

In one embodiment, an isolated population of cells expressing STAT3 phosphorylated at serine 727 and nucleostemin is provided. In another embodiment, the isolated population of cells expressing STAT3 phosphorylated at serine 727 and nestin, Hes3 and/or sonic hedgehog (SHH) is provided. In a further specific, non-limiting example the cells are STAT3Ser727⁺nestin⁺SHH⁺Nucl⁺ cells.

Generally, the cells are mammalian cells. In one specific, non-limiting example the cells are murine cells. In other specific, non-limiting example the cells are non-human primate or human cells. Compositions including these cells are also provided.

Any method known to one of skill in the art can be used to isolate the cells, or populations of cells. In one embodiment, suspension of cells including stem cells and/or progenitor cells is produced. In one embodiment, embryonic stem cells or neuronal precursor cells are utilized as a starting population of cells. In one example, a human embryonic stem cell line is utilized.

Antibodies that specifically bind a cell surface marker of interest, such as CD133 and/or STAT3, are then reacted with the cells in suspension. Methods of determining the presence or absence of a cell surface markers are well known in the art. Typically, labeled antibodies specifically directed to the marker are used to identify the cell population. The antibodies can be conjugated to other compounds including, but not limited to, enzymes, magnetic beads, colloidal magnetic beads, haptens, fluorochromes, metal compounds, radioactive compounds or drugs. The enzymes that can be conjugated to the antibodies include, but are not limited to, alkaline phosphatase, peroxidase, urease and β-galactosidase. The fluorochromes that can be conjugated to the antibodies include, but are not limited to, fluorescein isothiocyanate, tetramethylrhodamine isothiocyanate, phycoerythrin, allophycocyanins and Texas Red. For additional fluorochromes that can be conjugated to antibodies see Haugland, R. P., Molecular Probes: Handbook of Fluorescent Probes and Research Chemicals (1992-1994). The metal compounds that can be conjugated to the antibodies include, but are not limited to, ferritin, colloidal gold, and particularly, colloidal superparamagnetic beads. The haptens that can be conjugated to the antibodies include, but are not limited to, biotin, digoxigenin, oxazalone, and nitrophenol. The radioactive compounds that can be conjugated or incorporated into the antibodies are known to the art, and include but are not limited to technetium 99m (⁹⁹Tc), ¹²⁵I and amino acids comprising any radionuclides, including, but not limited to, ¹⁴C, ³H and ³⁵S.

Fluorescence activated cell sorting (FACS) can be used to sort cells that express a marker of interest, such as but not limited to AC133, by contacting the cells with an appropriately labeled antibody. In one embodiment, additional antibodies and FACS sorting can further be used to produce substantially purified populations of cells.

A FACS employs a plurality of color channels, low angle and obtuse light-scattering detection channels, and impedance channels, among other more sophisticated levels of detection, to separate or sort cells. Any FACS technique may be employed as long as it is not detrimental to the viability of the desired cells. (For exemplary methods of FACS see U.S. Pat. No. 5,061,620, herein incorporated by reference).

However, other techniques of differing efficacy may be employed to purify and isolate desired populations of cells. The separation techniques employed should maximize the retention of viability of the fraction of the cells to be collected. The particular technique employed will, of course, depend upon the efficiency of separation, cytotoxicity of the method, the ease and speed of separation, and what equipment and/or technical skill is required.

Separation procedures may include magnetic separation, using antibody-coated magnetic beads, affinity chromatography, cytotoxic agents, either joined to a monoclonal antibody or used in conjunction with complement, and “panning,” which utilizes a monoclonal antibody attached to a solid matrix, or another convenient technique. Antibodies attached to magnetic beads and other solid matrices, such as agarose beads, polystyrene beads, hollow fiber membranes and plastic petri dishes, allow for direct separation. Cells that are bound by the antibody can be removed from the cell suspension by simply physically separating the solid support from the cell suspension. The exact conditions and duration of incubation of the cells with the solid phase-linked antibodies will depend upon several factors specific to the system employed. The selection of appropriate conditions, however, is well within the skill in the art.

The unbound cells then can be eluted or washed away with physiologic buffer after sufficient time has been allowed for the cells expressing a marker of interest to bind to the solid-phase linked antibodies. The bound cells are then separated from the solid phase by any appropriate method, depending mainly upon the nature of the solid phase and the antibody employed.

Antibodies may be conjugated to biotin, which then can be removed with avidin or streptavidin bound to a support, or fluorochromes, which can be used with a fluorescence activated cell sorter (FACS), to enable cell separation (see above).

Release of the cells from the magnetic beads can effected by culture release or other methods. Purity of the isolated cells is then checked with a FACSCAN® flow cytometer (Becton Dickinson, San Jose, Calif.), for example, if so desired. In one embodiment, further purification steps are performed, such as FACS sorting the population of cells released from the magnetic beads.

In one embodiment, magnetic bead separation is used to first separate a population of cells that do not express more than one lineage specific markers. In addition, panning can be used to separate cells that do not express one or more lineage specific markers (for panning methods see Small et al., J Immunol Methods 3; 167(1-2):103-7, 1994, herein incorporated by reference). These cells can then be removed, and the population of interest further purified.

The disclosure is illustrated by the following non-limiting Examples.

### EXAMPLES

### Example 1

### Material and Methods

Reagents: The following compounds were used in the experiment described below: FGF2 (233-FB), Jagged-1 (599-JG), Delta-4 (1389-D4), CNTF (577-NT), Fibronectin (1030-FN), from R&D; JAK Inhibitor I (420099), DAPT (565770), LY294002 (440204), SU6656 (572636), KN92 (422709), KN93 (422708), KN62 (422706), rapamycin (553210), SB203580 (559389), from Calbiochem. Also, Polyornithine (Sigma, P-3655), human LIF (Chemicon, LIF1005), ECL reagents (Pierce, 34080), polyacrylamide gradient gels (Invitrogen), BrdU (Boehringer, 84447723), Goat anti-human Fc (Jackson Immunoresearch), Alexa-Fluor-conjugated secondary antibodies (Molecular Probes), HRP-conjugated secondary antibodies (Jackson Immunoresearch), DAPI (Sigma, D-8417), and general chemicals from Sigma.

Cell culture: E13.5 cortical embryonic mouse and adult rat subventricular zone CNS stem cells were grown as previously described (Johe et al., Genes Dev 10:3129-40, 1996; Rajan et al., J Neurosci 18:3620-9, 1998). Cells were expanded in serum-free DMEM/F12 medium with N2 supplement with FGF2 (20 ng/ml) for 5 days and were re-plated at 1000-10,000 cells per cm². FGF2 was included throughout our experiments, unless otherwise stated.

The human ES cell lines HSF-6 (University of California, San Francisco; NIH # UC06, Passage 38-120) and H1 and H9 (WiCell Research Institute, NIH # WA01 and WA07) were maintained on mouse embryonic fibroblasts (MEF) according to the suppliers' protocols. Immunocytochemistry and FACS analysis for the expression of Oct4 and SSEA4 and SKY karyotyping established that the starting cells are undifferentiated and have a normal set of chromosomes. For single cell survival experiments, hES colonies were harvested after treatment with 1.5 mg/ml collagenase type IV (Invitrogen), separated from the MEF feeder cells by repeated washing by gravity, gently triturated to break up colonies into small aggregates, further dissociated into a single cell solution by trypsination (0.05% trypsin/0.04% EDTA) and transferred on MEF feeder cells at a density of 10000 cells/ml. Oct-4 positive colonies were counted six days after plating. For experiments under feeder free culture conditions, collagenase harvested cell aggregates after separation from MEF cells were transferred to laminin (50 μg/ml) coated dishes in MEF condition medium (CM) as described (Xu et al., Nat Biotechnol 19:971-4, 2001). The medium was changed 24 hours later to either CM or normal unconditioned human ES cell culture medium. To induce differentiation into neural precursor cells and dopaminergic neurons, collagenase harvested hES cell aggregates after separation from MEF cells were transferred non-adherent suspension culture dishes (Corning) in human Es cell medium in the presence of 50 ng/ml FGF4. After eight days in suspension, human embryoid bodies were plated onto adherent tissue culture dishes in ITSFn Medium (Kim et al., Nature 418:50-6, 2002; Lee et al., Nat Biotechnol 18:675-9, 2000; Okabe et al., Mech Dev 59:89-102, 1996) supplemented with 5 μg/ml fibronectin. After eight days of selection of nestin positive precursors, cells were dissociated by trypsination, and plated on poly-L-ornithine-coated (15 μg/ml) tissue culture plates at a density of 2×10⁵ cells/cm² in N2 Medium (Kim et al., Nature 418:50-6, 2002; Lee et al., Nat Biotechnol 18:675-9, 2000; Okabe et al., Mech Dev 59:89-102, 1996) medium containing 20 ng/ml FGF2, 500 ng/ml SHH and 100 ng/ml FGF8. After six days of expansion of nestin positive cells, medium was replaced by N2 medium without growth factors to induce terminally differentiation into neurons.

mES (D3) cells were obtained from ATCC and were maintained in feeder-free cultures on gelatin-coated plates supplemented with 1400 units/ml LIF.

Immunofluorescent staining of cells: Immunocytochemistry was performed as previously described (Cameron et al., Nat Neurosci 2:894-7, 1999; Panchision et al., Genes Dev 15:2094-110, 2001). Antibodies were used that specifically bind: Notch1IC (Chemicon, 5352), GFP (Molecular Probes, A11122), Oct3/4 (Santa Cruz, sc-5279), SSEA1 and SSEA4 (Developmental Studies HybridomaBank), Tra-1-60 (Chemicon, MAB4360), Tra-1-81 (Chemicon, MAB4381), human nestin (Chemicon, MAB5326), Sox-1 (Gift from Dr. R Lovell-Badge), TH (Pel-Freez, P40101-0), Tuj1 (Covance, MMS-435P), rat nestin (McKay Lab), GFAP (Dako, z0334), CNPase (Chemicon, MAB326), BrdU (Accurate, H5903), DCX (Santa Cruz, sc-8066), Sox2 (R&D, MAB2018), Hu (Molecular Probes, A21271).

Immunoblotting: Immunoblotting was performed as previously described (Panchision et al., Genes Dev 15:2094-110, 2001). The following antibodies were used in the experiments described below: pSer473-Akt (92715), pSer308-Akt (9275), pSer2448-mTOR (29715), pThr180/Tyr182-p38 (9211), pSMAD1/5/8 (9511) from Cell Signaling; STAT3 (482), pSer727-STAT3 (8001-R), pTyr705-STAT3 (7993) from Santa Cruz; Sonic Hedgehog (R&D, AF464), nucleostemin (McKay Lab), Hes3 (Chemicon, AB5706), α-tubulin (Sigma, T-6074).

RT-PCR: For reverse-transcriptase PCR analysis, RNA was extracted from cell cultures or brain homogenates with Trizol (Invitrogen, 15596-026), and PCR reactions were performed with the ProSTAR First-Strand RT-PCR Kit (Stratagene, 200420). Primers were used for the following genes: Hes1, Hes2, Hes3, Hes5, Hes6, Hey2; Hes7

| | |
|---|---|
| Hes7 | |
| (sense, | 5′-GCTCGCCAGCTGCTACTTGT-3′; |
| antisense, | 5′-AGCAGTGGGATGGGGACCAA-3′), |
| HeyL | |
| (sense, | 5′-GGTCTCTGTGCAGGCCTGTA-3′; |
| antisense, | 5′-CAGGGGACTTGAGTTCTCAG-3′), |
| Hes3b | |
| (sense, | 5′-CCAGCCAGCAGCTTCCGAAA-3′; |
| antisense, | 5′-CATCGGTGGAAGACTCAAGGAG-3′). |

Nucleofection: For overexpression of plasmid DNA in CNS SC the Amaxa nucleofector kit (VPG-1004) was used. Five million cells were mixed with 2 μg plasmid DNA per reaction. The plasmids used were (Kitamura et al., Exp Hematol 31:1007-14, 2003) (pMX-STAT3 (WT)-IRES-GFP, pMXs-STAT3 (Y705F)-IRES-GFP, pMXs-STAT3 (S727A)-IRES-GFP).

In-vivo Experiments: All drugs were infused over one week using an Alzet osmotic pump (model 2001 delivering at a rate of 1 μl/hr for seven days). Briefly, an intracerebral cannula connected to the pump was inserted into the right lateral ventricle using the following stereotaxic coordinates: bregma AP +0, Lateral −1.5 mm dorso-ventral 3.3 mm. Animals received IP injection of the tracer BrdU (50 mg/kg) every 12 hours for 5 days beginning on day one post-operatively to label dividing cells.

For the induction of focal ischemia in rats, male spontaneously hypertensive rats (SHR) underwent permanent middle cerebral artery occlusion (PMCAO) (Leker et al., Stroke 33:1085-92, 2002). This is a highly reproducible model of focal irreversible cerebral ischemia that results in isolated fronto-parietal cortical injury. For motor disability evaluations, animals were analyzed with a standard motor disability protocol (Leker et al., Stroke 34:2000-6, 2003).

Immuno-positive cells were analyzed by confocal microscopy counted using a non-biased system on slides obtained from homologous coronal slices containing the infarct of each animal at the position between +0.2 and −3.8 mm from the center of the bregma according to stereotaxic coordinates. Cells were enumerated in thirteen regions of interest, on both the ipsilateral and contralateral hemispheres (to cannula, or cannula and ischemic focus): subventricular zone (SVZ), corpus callosum and white matter and the peri-infarct cortex.

Statistical Analysis: Analysis was performed with the SigmaStat package (SPSS inc. Richmond Calif.). Data are presented as mean ±SD or mean ±SEM as indicated in the legends. Values were compared using analysis of variance with Dunette's test and analysis of variance on repeated measures as indicated in the legends.

### Example 2

### Notch Ligands and p38 Inhibitors Stimulate Survival in CNS Stem Cells

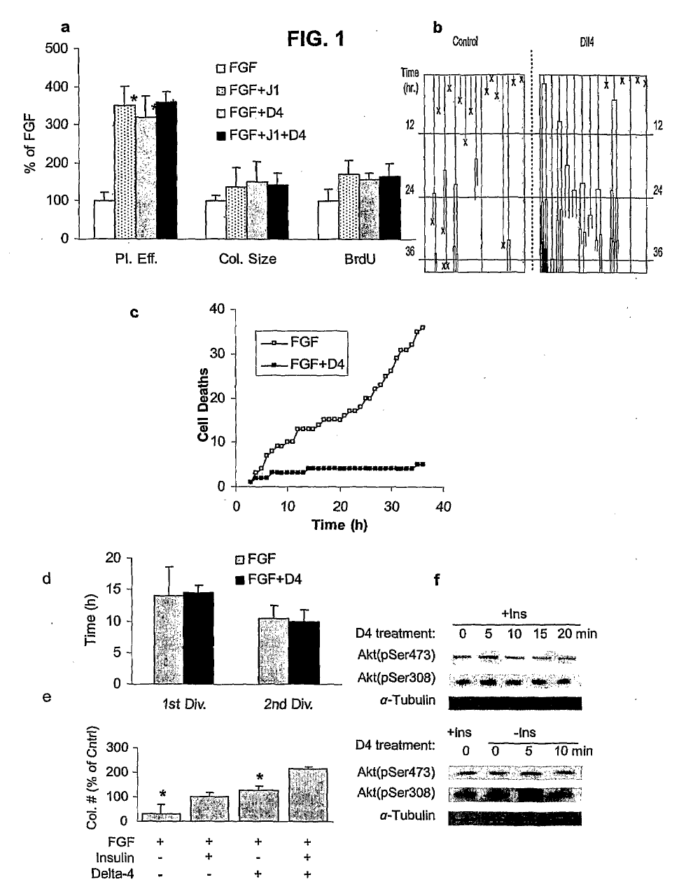

In the CNS, in vivo gene deletion studies have implicated Notch in self renewal, whereas in vitro studies suggest that Notch promotes developmental progression to astrocytic fates (Zhong et al., Development 124:1887-97, 1997; Morrison et al., Cell 101:499-510, 2000; Tanigaki et al., Neuron 29:45-55, 2001; Kamakura et al., Nat Cell Biol 6:547-54, 2004). To explore these paradoxical observations, CNS stem cell cultures from mid-gestation (embryonic day 13.5) mouse dorsal telencephalon were treated with soluble Notch ligands. Clonal analysis shows that these cells can generate the three major cell types of the CNS (Johe et al., Genes Dev 10:3129-40, 1996). Following initial passaging, the cells were plated at a density of 1000 cells/cm², cultured for 5 days in the presence of FGF2, and colony numbers, sizes, and BrdU incorporation (after a 4 hour BrdU pulse) were enumerated. Notch activation by Jagged-1 or Delta-4 (200 ng/ml) increased the number of colonies generated in vitro by over 3-fold (FIG. 1a). The average colony size and proportion of BrdU+ cells did not significantly increase. The effects of Notch activation were most dramatic when treatments were initiated at the start of the culture. This result suggests that Notch acts as a pro-survival signal that is effective when the cells are passaged.

To assay for Notch activation, cultures were treated with the Notch ligands for 1 hour and Notch1 activation was detected by immunocytochemical staining of Notch1IC (FIG. 1b). Notch activation is dependent on a proteolytic cleavage by γ-secretase and the inhibitor DAPT blocks the accumulation of Notch1IC in these cells.

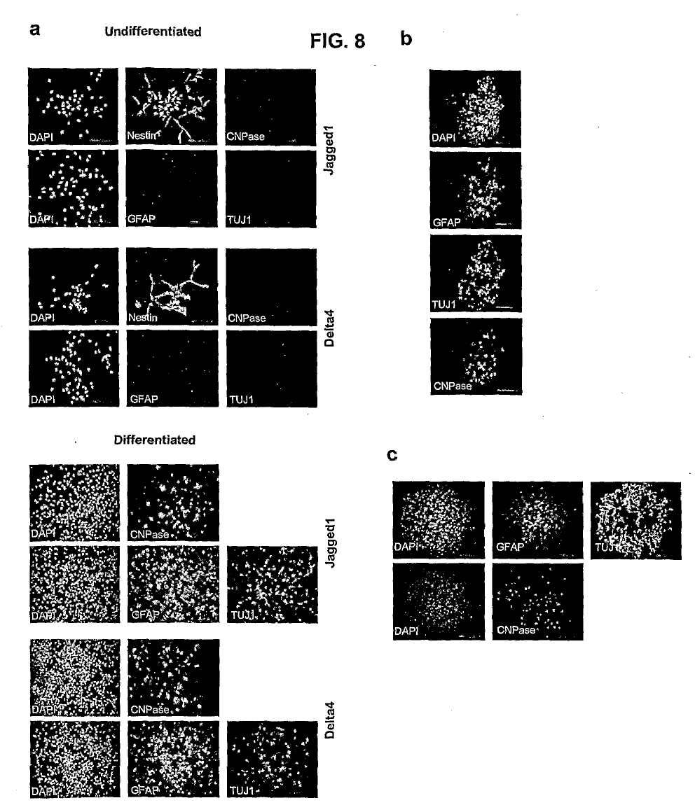

To test whether Notch activation promoted differentiation or fate commitment, these cultures were treated with either Jagged-1 or Delta-4 for 5 days, at which point the cultures were fixed or allowed to differentiate by FGF2 and Notch ligand withdrawal for an additional five days. The proportion of cells expressing genes characteristic of the stem or differentiated cells was determined (FIG. 8A). The results show that treatment with either Notch ligand retained the morphology and antigen profile of stem cells, and that these cells were competent to generate the three dominant fates of the central nervous system (CNS) (neurons, astrocytes, and oligodendrocytes) in the same ratios seen in control cultures (see Table 3).

TABLE 3
Notch ligands do not alter the differentiation potential
of embryonic CNS stem cells*

| | % Neurons | % Glia | % Oligodendrocytes |
|---|---|---|---|
| | (TUJ1) | (GFAP) | (CNPase) |
| FGF [symbol] WD | 42.37 ± 6.85 | 49.53 ± 4.79 | 9.12 ± 2.82 |
| FGF + D4 [symbol] WD | 44.27 ± 9.83 | 47.02 ± 5.15 | 7.82 ± 4.32 |
| FGF + J1 [symbol] WD | 41.15 ± 11.1 | 48.84 ± 6.97 | 8.14 ± 5.8 |
| FGF + D4 + J1 | 43.23 ± 4.63 | 45.08 ± 6.89 | 8.77 ± 4.45 |
| [symbol] WD | | | |

*E13.5 cortical stem cells were treated with Jagged-1, Delta-4, and combination in the presence of bFGF2 (20 ng/ml) for 5 days and were subsequently instructed to differentiate by mitogen withdrawal (WD) for 3 days. Control cells were not treated with Notch ligands. The ratios of astrocytes, neurons, and oligodendrocytes obtained by all treatments were comparable.

The multipotentiality of treated cells was tested more rigorously by plating them at clonal density (50 cells/cm²). Following five days of Delta-4+FGF2 treatment and 5 days of differentiation, 65% of the clones contained GFAP+, TUJ1+, and CNPase+ cells (FIG. 8B).

These data indicated that Notch ligands do not cause changes in fate but improve the plating efficiency of CNS stem cells. To define this effect at higher resolution, the fates of individual cells were followed over a period of 36 hours from plating with a real-time imaging system. Examples of lineage analysis in the presence or absence of Delta-4 are shown in FIG. 1b. There was cell death immediately after plating in both conditions but 4 hours after plating, very few death events were observed in Delta-4—treated cultures, compared to controls (FIGS. 1b and 1c). Most cells divided two or three times during the observation period (FIG. 1c) and in both control and Delta-4 treatment conditions, the first cell cycle lasted approximately 14 hours (14.08 h±4.6 for FGF2 and 14.49 h±2.0 for FGF2+Delta-4) and the second approximately 10 hours (10.44 h±1.18 for FGF2 and 9.89 h±1.91 for FGF2+Delta-4). These data show that Delta-4 treatment had a clear effect on cell survival without affecting the cell cycle.

Insulin in the medium is also thought to promote cell survival. Given the results suggesting that Notch also promotes survival, it was tested whether Notch receptor activation could compensate for insulin omission. Seeding cells in the absence of insulin caused the plating efficiency measured at five days to drop to 29.9%±37.9 (FIG. 1e). Delta-4 was able to compensate for the lack of insulin in the medium, and restored plating efficiency (125%±19.4). Because Notch activation improved survival, activation of Akt, a ser/threonine kinase known to be involved in cell survival, was assayed. Notch activation rapidly (5 minutes) promoted the phosphorylation of Akt on Ser473 and Ser308 in both the presence and absence of insulin (FIG. 1f). These results show that both Notch and insulin promote Akt activation and survival of CNS stem cells.

### Example 3

### Notch Ligands Stimulate Phosphorylation of Ser727 on STAT3

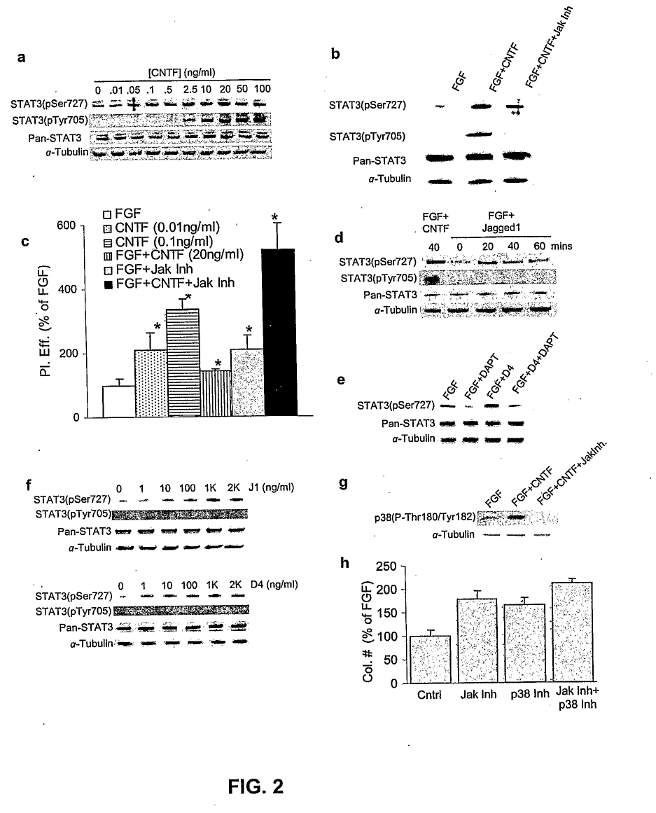

Ligands that activate JAK/STAT signaling promote astrocytic differentiation of CNS stem cells (Johe et al., Genes Dev 10:3129-40, 1996; Raj an et al., J Neurosci 18:3620-9, 1998; Bonni et al., Science 278:477-83, 1997; Song et al., Nat Neurosci 7:229-35, 2004; He et al., Nat Neurosci 8:616-625, 2005). When applying CNTF to CNS stem cells it was noted that low concentrations of CNTF stimulated survival but not expression of astrocyte specific genes. This is consistent with previous observations of Akt phosphorylation downstream of gp130 activation (Taga et al., Annu Rev Immunol 15:797-819, 1997). A dose response curve shows that low concentrations (<0.5 ng/ml) of CNTF induce the phosphorylation of STAT3 on Ser727 while phosphorylation on Tyr705 only occurs at higher doses (>1 ng/ml; FIG. 2A). At high concentrations (20 ng/ml) the phosphorylation of Tyr705 can be blocked by an inhibitor of JAK while allowing a CNTF-induced increase in Ser727 phosphorylation (FIG. 2B) (Thompson et al., Bioorg Med Chem Lett 12:1219-23, 2002). Low doses of CNTF or high doses in the presence of the JAK inhibitor support CNS stem cell survival (FIG. 2C). These results show STAT3 Ser727 phosphorylation correlates with cell survival.

Notch activation also increased the phosphorylation of STAT3 on serine 727, but not on tyrosine 705 (FIG. 2d). CNTF was used as a positive control; CNTF, induced the phosphorylation on both sites. With both CNTF and Notch activation, phosphorylation peaked at around 30 min. STAT3 phosphorylation on Ser727 following Notch activation was sensitive to the γ-secretase inhibitor DAPT (FIG. 2e). DAPT reduced the basal levels of pSer727 suggesting that endogenous Notch activity is partly responsible for these basal levels. Dose response experiments for Jagged-1 and Delta-4 found that peak STAT3 phosphorylation was achieved at approximately 10 ng/ml for Jagged-1 and 1 ng/ml for Delta-4 (FIG. 2f). In contrast to CNTF, no Tyr705 phosphorylation was detected at any of the doses of Notch receptor ligands.

These results suggest that STAT phosphorylation on Ser727 could mediate the cell survival effects of both Notch ligands and CNTF. Because JAK inhibition increased survival when no Tyr705 signal was detected (FIG. 2c), it was hypothesized that JAK inhibition could promote proliferation both by inhibiting Tyr705 and through another target. A candidate was p38MAP kinase, known to act downstream of JAK in many cells including CNS stem cells. p38 kinase has been implicated both in differentiation and cell cycle regulation, and recently, inhibition of p38 kinase enabled the proliferation of adult mammalian cardiomycetes (Engel et al., Genes Dev, 2005). Inhibition of JAK blocked CNTF induced phosphorylation of p38 (FIG. 2g). The p38 inhibitor enhanced survival to a similar extent to the JAK inhibitor (FIG. 2h). Combined JAK and p38 inhibition did not improve survival further. These data suggest that a JAK/p38 pathway promotes cell death and a pathway activated by Notch ligands and low concentrations of CNTF promotes cell survival through phosphorylation of Ser727 in STAT3.

### Example 4

### Serine727 on STAT3 Integrates Second Messenger Pathways that Control Survival

Several serine/threonine kinases have been proposed as direct or indirect mediators of STAT3 phosphorylation. Given the finding that Akt is activated in response to Notch, it was tested whether PI3kinase, a critical regulator of Akt activity is important for Notch-induced STAT3 phosphorylation. STAT3 phosphorylation downstream of Notch activation was sensitive to the PI3 Kinase inhibitor LY294002 (FIG. 3a). Src Kinase is thought to act downstream of Akt and to be essential for cell survival in many cell types including human embryonic stem (hES) cells (Zhao et al., Biochem Biophys Res Commun 325:541-8, 2004). The small molecule inhibitor of Src, SU6656, blocked the regulatory effect of Notch activation on STAT3 pSer727 (FIG. 3b).

It has been demonstrated that mTOR activity was required for BMP-mediated phosphorylation of STAT3 on Ser727, and that mTOR and STAT3 could be co-immunoprecipitated (Rajan et al., J Cell Biol 161:911-21, 2003). Notch activation promoted the phosphorylation of mTOR on Ser2448 (FIG. 3c), with a time-course similar to that for STAT3 phosphorylation (FIG. 2d). The specific mTOR inhibitor rapamycin blocked STAT3 phosphorylation suggesting that mTOR is an essential mediator of STAT3 phosphorylation following Notch activation (FIG. 3d).

Cam Kinase II has also been implicated in STAT3 phosphorylation (Nair et al., Proc Natl Acad Sci USA 99:5971-6, 2002). Two Cam Kinase II inhibitors, KN93 and KN62 had a similar inhibitory effect, whereas the negative control KN92 had no effect on STAT3 phosphorylation (FIG. 3e). These results suggest that mTOR and Cam Kinase II are required for STAT3 phosphorylation following Notch activation. Consistent with a role of STAT3-Ser727 phosphorylation in survival, a five-day treatment with various inhibitors of this phosphorylation were strongly cytotoxic (FIG. 3f).

The Notch induced activation of STAT3 and mTOR is transient with a peak at around 30 minutes followed by an abrupt down-regulation (FIGS. 2d and 3c). LKB1 is a known inhibitor of mTOR as well as GSK3β. Therefore, the role of LKB1 in the observed decrease in STAT3 and mTOR phosphorylation was examined. LKB1 itself is regulated by MSK-1 kinase, and it was investigated whether mTOR inactivation followed MSK-1 and LKB1 activation. Notch activation promoted the activating phosphorylation of LKB1 and of MSK-1, a kinase known to be involved in LKB1 activation. Inactivating phosphorylation of GSK-3 and an activating de-phosphorylation of β-catenin also occurred following Notch activation (FIG. 3g). These data suggest that the Notch receptor promotes both positive and negative effects that regulate the duration and levels of mTOR and STAT3 activation.

The signaling and pharmacological data suggest that post-translational modification of the Ser727 in STAT3 regulates stem cell survival. A genetic approach was used to allow an independent technical assessment of the central role of STAT3 in cell survival pathways. CNS stem cells were transfected with wild-type STAT3 and two mutant forms where either Tyr705 or Ser727 were altered to ‘neutral’ amino acids (STAT3-YF and STAT3-SA). In addition to STAT3, these plasmids contained an IRES controlling expression of the green fluorescent protein (GFP) allowing the number of transfected cells to be measured at 24 hours and 4 days after transfection. Transfection efficiency was similar with all three cDNAs (˜50% GFP+ cells at 24 h). In contrast, at 4 days the number of wild-type and STAT3-YF transfected cells was unchanged (STAT3 wt, 100%±129.31; STAT3-YF, 100%±27.45) but the proportion of STAT3-SA transfected cells was greatly reduced (16%±11.14; FIGS. 3h, 3i). Similar results were obtained when the transfection experiment was performed in the presence of Delta-4 and the JAK inhibitor (STAT3 wt, 100%±29.17; STAT3-YF, 109%±23.40; STAT3-SA, 20.79%±3.79). These data show that cells can tolerate altered levels of wild-type STAT3 or STAT3 lacking Tyr705 but STAT3 lacking Ser727 is acutely cytotoxic. These data using genetic manipulation confirm that phosphorylation of STAT3 on the serine727 is essential for survival.

### Example 5

### Downstream Stat3-Ser Effectors

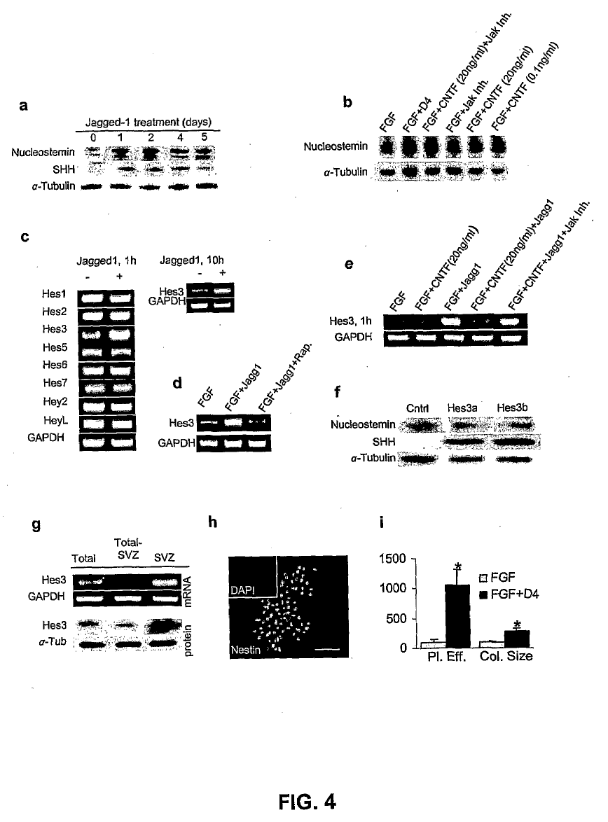

The interest in stem cell self-renewal mechanisms prompted analysis of potential down-stream components of the Notch/STAT3-Ser727 (Notch/sSTAT) pathway. Nucleostemin is a p53-binding protein expressed in self-renewing CNS stem cells and cancer cells (Tsai et al., J Neurosci 20:3725-35, 2000; Tsai et al., J Cell Biol 168:179-84, 2005). Sonic Hedgehog (SHH) is a protein with functions in fate specification and stem cell survival in the fetal and adult CNS (Palma et al., Development 132:335-44, 2005). Notch activation by Jagged1 increased both nucleostemin and SHH protein expression and both proteins were present at elevated levels for days following transfection (FIG. 4a). Nucleostemin levels were also increased by low doses of CNTF or higher doses in the presence of the JAK Inhibitor (FIG. 4b). These data show that Ser727 phosphorylation at 30 minutes after Notch activation predict nucleostemin levels at 48-72 hours. These results suggest that the Notch/sStat3 pathway can activate intermediate-term responses that sustain stem cell survival.

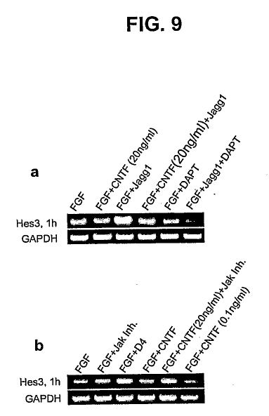

Notch signaling regulates transcription of the Hes/Hey gene family, a group of genes implicated in the control of differentiation (Iso et al., J Cell Physiol 194:237-55, 2003). RT-PCR was used to assess mRNA regulation of these genes one hour after Notch activation in CNS stem cells. Only Hes3 mRNA levels were significantly altered at 1 hour (FIG. 4c). The Hes3 mRNA remained elevated 10 hours after stimulation and was sensitive to rapamycin suggesting that mTOR activation in the first hour of stimulation was responsible for elevated transcription (FIG. 4d). Both the γ-secretase inhibitor DAPT (FIG. 9a) and high CNTF concentrations (>1 ng/ml) inhibited Hes3 induction by Jagged1 (FIG. 4e). The ability of high concentrations of CNTF to inhibit Hes3 induction following Notch activation was dependent on JAK activity (FIG. 4e). In contrast, in the absence of added Notch ligand, Hes3 mRNA levels were elevated by high CNTF and the JAK inhibitor (FIG. 4e). The data suggest that Hes3 mRNA levels are regulated by the Notch/sSTAT survival pathway. Treatment with the JAK inhibitor alone had no effect on Hes3 mRNA (FIG. 9b) but Hes3 mRNA levels were elevated under conditions that support increased levels of nucleostemin, SHH and STAT3 phosphorylation on Ser727.

Hes3 mRNA levels were elevated within an hour of Notch treatment suggesting that the slower changes in SHH and nucleostemin levels are downstream events. There are two forms of mRNA encoded by the Hes3 gene, Hes3a and Hes3b. CNS stem cells were transfected with Hes 3a and 3b cDNAs showed no increase in nucleostemin levels but both cDNAs gave a clear increase in SHH protein (FIG. 4f). Stem cells treated with SHH for 1 and 2 days showed no increase in nucleostemin protein expression. These results suggest that SHH is downstream of Hes3 but that the nucleostemin pathway is independently controlled by the upstream Notch/STAT3-Ser727 components.

### Example 6

### Notch Activation Increases the Generation of Adult CNS Stem Cells In Vitro

The data presented herein show that the Notch/sSTAT pathway increases Hes3 transcription and promotes survival in fetal CNS stem cells in vitro. The pattern of Hes3 mRNA expression has been shown by whole embryo in situ hybridization to peak at around embryonic day 9.5 and to be absent a few days later (Hirata et al., Embo J 20:4454-66, 2001). This pattern is similar to the expression of nestin, an intermediate filament protein expressed in CNS stem cells in the fetal and adult nervous system (Frederiksen et al., J Neurosci 8:1144-51, 1988; Reynolds et al., Science 255:1707-10, 1992). The in situ method may not be sensitive enough to detect Hes3 in the small numbers of adult CNS stem cells.

RT-PCR and Western blotting was used to show that Hes3 mRNA and protein were both expressed in the subventricular zone (SVZ), one of two major neurogenic regions of the adult brain. Very little protein or mRNA were detected outside the SVZ (FIG. 4g). Adult CNS stem cells can be cultured as a monolayer in vitro (FIG. 4h) and they show similar responses to extracellular signals that regulate stem cell differentiation upon withdrawal of the mitogen that sustains the undifferentiated state (Johe et al., Genes Dev 10:3129-40, 1996). To ask if Notch activation promotes survival in rat adult SVZ cultures, isolated cells were grown in the presence of FGF2 with and without Delta-4 for one week. Plating efficiency was greatly increased (1050%±273.8), in the presence of FGF2 for one week (FIG. 4i). In contrast to the effects on fetal CNS stem cells, treatment with Delta-4 also increased colony size (278.5%±52.5; FIG. 4i). JAK inhibition modestly enhanced plating efficiency (approx. 40%). When FGF2 was withdrawn for a week, cells at clonal density generated all three CNS lineages (FIG. 8C, Table 4).

TABLE 4
Adult CNS stem cells retain multi-potentiality after Delta-4 treatment*

| | % Neurons | % Glia | % Oligodendrocytes |
|---|---|---|---|
| | (TUJ1) | (GFAP) | (CNPase) |
| FGF [symbol] WD | 43.3 ± 10.7 | 51.8 ± 10.3 | 9.4 ± 2.6 |

*Adult SVZ CNS SC were treated with Delta-4 in the presence of FGF2 (20 ng/ml) for 7 days and were subsequently instructed to differentiate by mitogen withdrawal for an additional 7 days. The ratios of astrocytes, neurons, and oligodendrocytes obtained from the two populations of cells expanded are shown.

These results show that Delta-4 treatment was consistent with expansion of adult CNS stem cells and raise the question of whether Notch activation affects plating efficiency of adult CNS stem cells. These data show that adult rat SVZ stem cells, like mouse embryonic CNS and hES cells respond to ligand-induced Notch activation and JAK inhibition with increased survival.

### Example 7

### Notch Activation and JAK Inhibition Promote Human ES Cell Survival

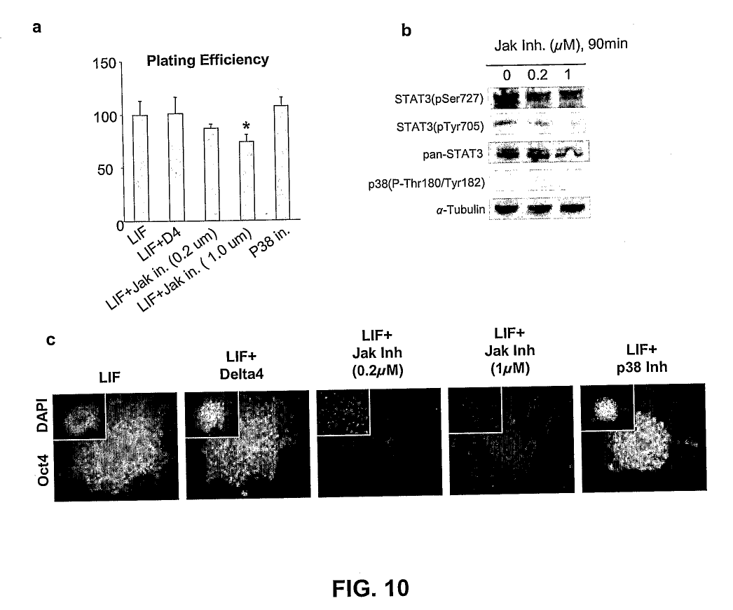

There is great interest in conditions that support the growth of mouse and human ES cells. In contrast to CNS SC (FIG. 2, FIG. 3) and hES SC (se below), the growth of undifferentiated mouse ES cells depends on the stimulation of the JAK/STAT pathway and STAT3 phosphorylation on Tyr705 has been specifically shown to be essential for self-renewal of mouse ES cells (Niwa et al., Genes Dev 12:2048-60, 1998; Matsuda et al., Embo J 18:4261-9, 1999). Ligands that stimulate the JAK/STAT pathway cause human ES cells to differentiate (Daheron et al., Stem Cells 22:770-8, 2004; Humphrey et al., Stem Cells 22:522-30, 2004). It was determined if the Notch/sSTAT survival pathway is present in mouse or human ES cells. It was found that JAK activity was essential for self-renewal of mES cells, and it was observed that JAK inhibition lead to a reduction in STAT3 phosphorylation not only on Tyr705, but also on Ser727, presumably due to the subsequent inhibition on p38 and MSK-1 kinases (FIGS. 10a-10c).

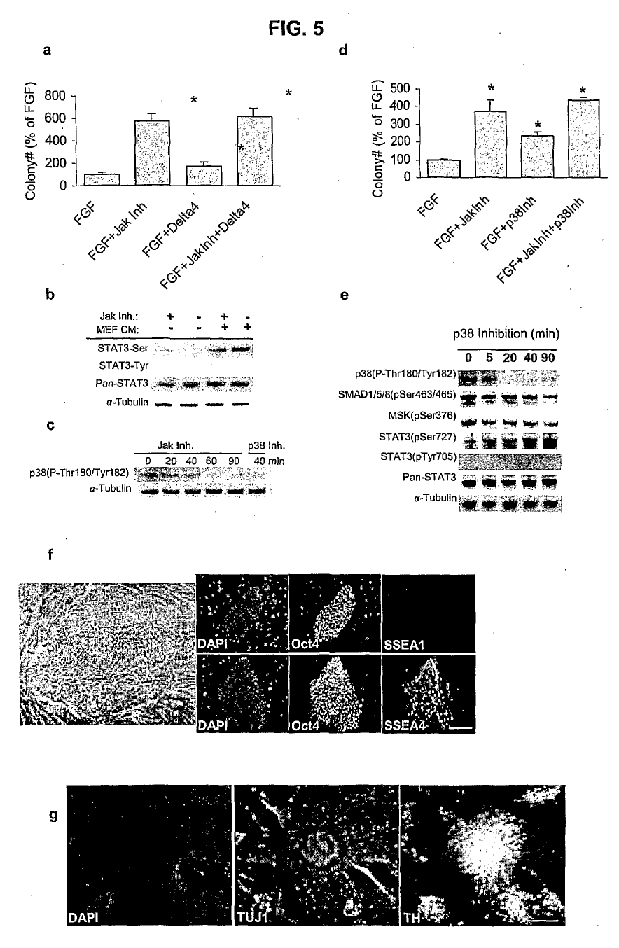

The pro-survival effects of activation of Notch and inhibition of JAK was tested on HSF-6 (NIH code UC06, http://escell.ucsf.edu), H1 (NIH code WA01, www.wicell.org) and H9 (NIH code WA09, www.wicell.org) hES cells. Singly dissociated hES cells were plated on mouse embryo fibroblasts (MEFs), treated daily for one week and colony numbers counted. Average colony sizes were not affected by stimulating Notch or inhibiting either JAK or p38 MAPK kinase. Plating efficiency was increased by all three treatments relative to FGF2 controls: FGF2, 100%±15.35; FGF2+JAK Inhibitor, 568.89%±70.65; FGF2+Delta-4, 172.155%±34.58; FGF2+JAK Inhibitor+Delta-4, 617.27%±76.57 (FIG. 5a). JAK inhibition also increased colony number formation of H1 and H9 lines (448.5%±57.4, 347.6%±59.5, respectively). These results show that both Notch ligands and the JAK inhibitor stimulate survival of hES cells.

In the absence of mouse fibroblast cells (MEF), hES cells can be maintained in the undifferentiated state by MEF-conditioned medium (CM). The effect of CM and JAK inhibition on STAT-3 phosphorylation was monitored. HSF6 hES cells were plated in CM for 24 hours, and the CM was withdrawn for another 16 hours, in the presence or absence of the JAK Inhibitor. Controls were kept in the presence of CM throughout the experiment. Tyrosine705 phosphorylation was undetectable in human ES cells and CM had a clear effect on Ser727 phosphorylation (FIG. 5b). The lack of Tyr705 phosphorylation and the presence of Ser727 phosphorylation suggest that CM activates a similar survival pathway in CNS stem cells and human ES cells.

The p38 MAPK is a target of JAK and inhibitors of JAK and p38 both stimulate CNS stem cell survival (FIGS. 2c and 2h). To determine whether in the hES system JAK inhibition reduced p38 activity, cells were treated in the absence of MEF with JAK inhibitor for different times and assayed for p38 phosphorylation. JAK inhibition significantly reduced p38 phosphorylation, within one hour (FIG. 5c). A one week treatment of hES cells with a p38 inhibitor also stimulated human ES cell survival (FIG. 5d).

Bone morphogenic proteins (BMPs) are members of the transforming growth factor (TGF)β superfamily and play critical roles in early development. Inhibition of BMP action is thought to be important in maintaining hES cells in the undifferentiated state. TGFβ family ligands inhibit growth in many cell types, including bone progenitors, and BMP action through the SMAD proteins is modulated by p38 activity (Abecassis et al., J Biol Chem 279:30474-9, 2004). The p38 MAPK and the Rho/ROCK pathways modulate SMAD function by stimulating inhibitory phosphorylation in the linker region of SMAD (Massague Genes Dev 17:2993-7, 2003). BMP inhibitors promote human ES cell growth (Pera et al., J Cell Sci 117:1269-80, 2004; Xu et al., Nat Methods 2:185-90, 2005). In hES cells treated with the p38 inhibitor, activating phosphorylation events on SMAD 1/5/8 were reduced in a time-dependent manner (FIG. 5e). p38 inhibition also reduced the phosphorylation of MSK-1 kinase, an activator of LKB1, and stimulated STAT3 phosphorylation on Ser727 with a similar time course. STAT3 phosphorylation on Tyr705 was absent in all conditions tested.

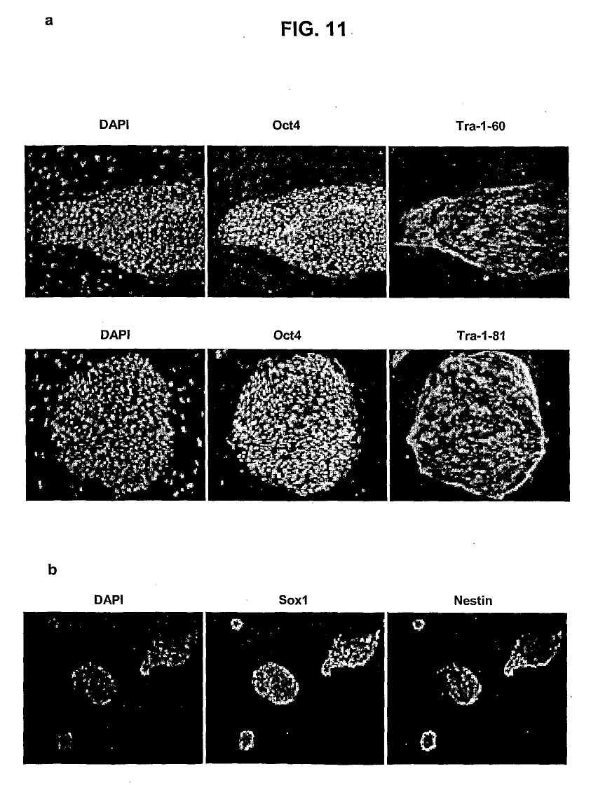

To assess whether JAK inhibition was consistent with self-renewal of hES cells, HSF6 cells were continuously exposed to the JAK inhibitor by daily additions for three passages as single cells (three weeks) and analyzed for expression of antigens that distinguish the undifferentiated and differentiated states. The cells exposed to the drug, like the FGF2 controls, had normal morphology, were positive for Oct3/4, SSEA4, Tra-1-60, and Tra-1-81, and negative for SSEA1 (FIG. 5f, FIG. 11). To assess their ability to differentiate into neural precursors, embryoid bodies (EBs) were generated and many Sox1+/Nestin+ cells obtained (FIG. 11). Finally, to assess that the cells retained their ability to generate TH+ neurons, direct differentiation protocols were performed; a high proportion of TH+/TUJ1+ cells were obtained (FIG. 5g). These results suggest that the action of BMP inhibitors Noggin and gremlin on hES cells (Pera et al., J Cell Sci 117:1269-80, 2004; Xu et al., Nat Methods 2:185-90, 2005) occurs through a similar Notch/sSTAT pathway that is present in CNS stem cells.

### Example 8

### The Survival of Pancreatic Precursors

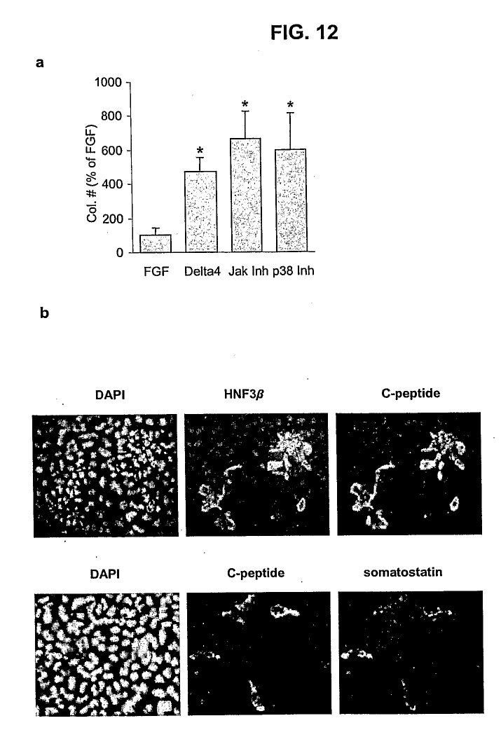

An increased generation of cells expressing markers of pancreatic islets was observed following Notch activation and inhibition of JAK and p38 kinases (FIG. 12A, 12B).

### Example 9

### Notch Increases the Generation of Adult CNS Stem Cells In Vivo

The effect of intraventricular administration of Delta-4 was studied in adult rats. As in other tissues, the numbers of stem cells in the adult CNS are precisely controlled. Previous work shows that delivery of FGF2 to the adult brain leads to an increased number of proliferating cells following stroke (Nakatomi et al., Cell 110:429-41, 2002). In vivo manipulations were designed to ask if Notch ligands can act alone or substantially alter the effect of FGF2 on the numbers of proliferating cells in the SVZ of the normal brain. Animals (N=5-6 per treatment) were fitted with a mini-osmotic pump administering artificial cerebrospinal fluid (ACSF, vehicle), FGF2, Delta-4, or a combination FGF2+Delta-4 for one week into the ventricles of three-month old rats. Animals were also given twice-daily injections of bromodeoxyuridine (BrdU) from post-op days two to six to label cells generated during this time window. Two groups of animals were analyzed at day 7 and day 45 relative to the initiation of protein delivery. Delta-4 doses were chosen to provide a roughly equal concentration in the rat adult cerebrospinal fluid as in the cell culture medium of our in vitro experiments.

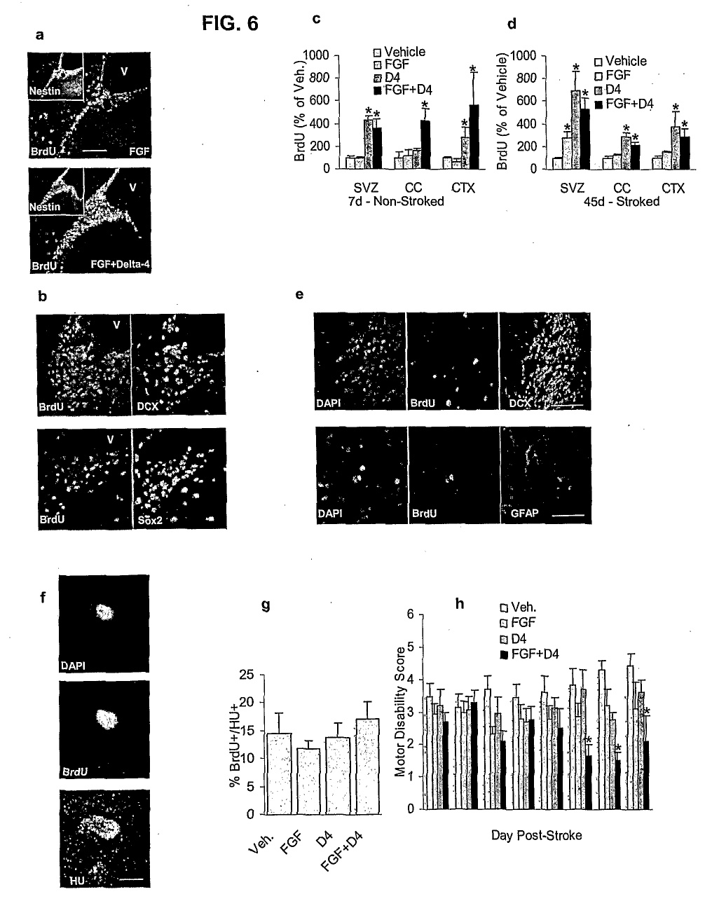

At day 7, FGF2, Delta-4, and combination treatments increased the number of BrdU labeled cells in the SVZ, corpus callosum (CC), and cortex (CTX) of the hemisphere ipsilateral to the cannula placement (which also acts as a point of local injury). Delta-4 treatment had a more potent effect than FGF2, and the combination treatment had the strongest effect. On the contralateral site, FGF2 infusion had no significant effect over vehicle on the number of proliferating cells. In contrast, Delta-4 treatment induced significant increases in BrdU incorporation in the contralateral SVZ and cerebral cortex (FIGS. 6a-c).

The combination of FGF2 and Delta-4 increased the numbers of BrdU labeled cells over vehicle control (Ipsilateral: 365%±75, SVZ; 422±105, cc; 570±288, cctx; Contralateral: 618±125, SVZ; 1126±370, cc; 2284±1832,cctx). A dose curve of Delta-4 (in the presence of a constant amount of FGF2 in the pump, 2.5 μg/ml) showed that maximal effects were achieved at approximately 0.42 μg/ml at the ipsilateral site and 4.2 μg/ml at the contralateral site. These data show that the combination of FGF2 and Delta-4 has a marked stimulatory effect over FGF2 alone on proliferating cells in the uninjured hemisphere. In all treatments, the new cells generated at the SVZ expressed the stem cell marker nestin, and the neuronal lineage marker doublecortin. A significant proportion (approx. 20%) of the BrdU+ cells also expressed the neural precursor marker Sox2 (FIG. 6b). These data show that delivery of the Notch ligand to the ventricle leads to a rapid increase in the number of cells that have recently proliferated. These cells express genes characteristic of stem cells and neuronal-restricted precursors.

At 45 days, Delta-4 and FGF2/Delta-4 treated animals also showed an increased number of BrdU+ cells in the SVZ, corpus callosum and the cerebral cortex relative to FGF2 treated animals. BrdU+ cells that were still at the SVZ co-expressed doublecortin (FIG. 6e). BrdU+ cells in the cortex were largely GFAP-negative (FIG. 6e) and rarely (<1%) expressed the neuronal markers NeuN and calretinin. However, many BrdU+ cells also expressed the early neuronal marker HU [13.95%±0.46, ipsilateral cerebral cortex; 10.2%±2.35, contralateral cerebral cortex; (FIGS. 6f,g)]. These data show that cells that proliferate during a transient exposure to the Notch ligands survive for long periods in the adult dorsal forebrain.

There is growing evidence for an endogenous repair process following injury to the telencephalon. A common model of clinical ischemia is achieved by occlusion of the middle cerebral artery (MCA) causing wide damage to the cerebral cortex and the underlying striatum. Two recent studies show that new neurons are not found in the cortex but are generated in the striatum following this injury (Arvidsson et al., Nat Med 8:963-70, 2002; Parent et al., Ann Neurol 52:802-13, 2002). If the MCA is cut at the surface of the brain, the subsequent damage is restricted to the overlying cerebral cortex (Leker et al., Stroke 33:1085-92, 2002). A series of simple motor tests is frequently used to assess the extent of injury in this model (Leker et al., Stroke 34:2000-6, 2003). After MCA occlusion by electrocoagulation at the pial surface, a canulla was placed to deliver growth factors to the ventricles in similar groups of animals as detailed above.

The size of the injury in the stroked animals was comparable in all treatment groups (21.3%±2.5, Vehicle; 21.0%±3.5, FGF2, 20.5%±2.9, FGF2+Delta-4; units: % of hemisphere volume). However, at 7 days post-treatment, combination Delta-4/FGF2 treatment generated more BrdU+ cells at the SVZ, corpus callosum, and cerebral cortex than FGF2 alone or vehicle, in both the ipsi- and the contra-lateral hemisphere. Treatment with Notch ligand with or without FGF2 increased the number of cells that were BrdU labeled in the days immediately following the injury and these cells survived for an additional 5 weeks (FIG. 6d). After FGF2 and Delta-4 treatment, there were few BrdU+ cells expressing astrocytic (GFAP) or mature neuronal (NeuN) markers. However, many BrdU+ cells expressed the antigens of immature neurons Hu and doublecortin (FIGS. 6e-g). The behavior of animals in the 45 day groups were assessed at the indicated time points. Combined treatment with FGF2/Delta-4 showed significant motor skill improvements starting at day 20 (FIG. 6h). These data suggest that ligand-induced Notch activation in vivo promotes the survival of newborn cells and diminishes the behavioral deficit caused by ischemia.

### Example 10

### Notch/Insulin Cross-Reactivity Identifies a Stem Cell Niche in the Brain

As described above, Notch activation in neural stem cells initiates a signal transduction cascade that shares many components with the insulin and classic cancer pathways and promotes their survival. Pharmacological Notch activation is rapidly followed by phosphorylation of Akt, a kinase downstream of insulin receptor activation with a central role in cancer biology. Following Akt activation, a key phosphorylation event on the serine residue 727 of STAT3 further mediates survival. It was investigated wherein the Notch and insulin pathways intercept.

It was determined that insulin also maintains phosphorylation of serine 727 in STAT3 in foetal neural stem cell cultures. Insulin can acutely induce (within 1 hour) phosphorylation of serine 727 of STAT3 if the cells are starved of insulin for two days, suggesting that insulin treatment activates key elements of the Notch signal transduction pathway and that STAT3-S727 is a mediator of well established survival pathways (Table 5).

TABLE 5
Insulin phosphorylates STAT3 on Ser727

| | +Ins, 2 days | −Ins, 2 days | −Ins, 2 days→ +Ins, 1 hour |
|---|---|---|---|
| STAT3-S727 | High | Low | High |
| STAT3-Y705 | No signal | No signal | No signal |

Conversely, Notch activation by treatment with the ligand Delta-like 4 (D114) induced rapid phosphorylation of Akt in the absence of insulin in the culture medium, suggesting that the Notch and insulin pathways integrate at the level of Akt activation (Table 6).

TABLE 6
Dll4 induces Akt phosphorylation in the absence of insulin

| | No Dll4 | Dll4, 5 min* | Dll4, 10 min |
|---|---|---|---|
| Akt-pS473 | Medium | High | Medium |
| Akt-pT308 | Medium | High | Medium |
| Akt | Medium | Medium | Medium |

min = minutes

The rapid integration of the Notch and insulin signals suggested that these pathways may intercept at the level of receptor activation. It was found that D114 treatment of foetal NSC cultures induced the rapid phosphorylation of the insulin receptor with kinetics similar to those of Akt activation (see Table 4). Specifically, D114 induced the phosphorylation of IGF1/insulin receptor. Peak phosphorylation occurred at 2-5 minutes.

Insulin treatment of the NSC cultures also induced cleavage (activation) of the Notch receptor (Table 7). It was determined that insulin facilitates Notch cleavage using immunocytochemical detection.

TABLE 7
Insulin and Dll4 facilitate each other in activating their receptors

| | | Insulin withdrawal, 2 days and then | | | | |
|---|---|---|---|---|---|---|
| | +Ins | treat for 1 h with: | | | | |
| | +Ins | −Ins | +Ins | Dll4 | DAPT + Ins | DAPT + Dll4 |
| Notch ICD | High | Low | High | Low | Low | Low |
| pIGF1 | High | Low | High | Low | Low | Low |
| (19H7) | | | | | | |
| pIGF1 | High | Low | High | Low | Low | Low |
| (3021) | | | | | | |

Insulin withdrawal for two days reduced the levels of cleaved Notch, and acute (1 hour) insulin but not D114 treatment induced Notch activation. The result suggested that insulin facilitates Notch activation by endogenous or externally added Notch ligands. Activation of the Notch receptor by addition of insulin or D114 was inhibited by DAPT, a γ-secretase inhibitor that blocks Notch cleavage.

As noted above, neural stem cell cultures express mRNA for the transcription factor Hes3; D114 treatment increases its transcription. Conversely, treatment with ciliary neurotrophic factor (CNTF), a cytokine that opposes the signal transduction pathway downstream of Notch activation opposed the ability of D114 to induce Hes3 transcription. Immunohistochemical experiments were performed that documented D114, CNTF regulate Hes3 expression. Thus, at the protein expression level, D114 maintains and CNTF inhibits Hes3 expression.

These results shows that the insulin and Notch pathways cross-react at the receptor level. In addition, the results demonstrate that insulin and Notch integrate at the level of second messenger activation. Hes3 expression is a suitable assay for these pro-survival pathways.

As there was a tight correlation between the Notch survival pathway and Hes3, it was investigated as to whether Hes3 is a mediator of the Notch pro-survival signalling. It was determined that both insulin and Notch activation promote the survival of foetal and adult neural stem cell cultures and their effects are additive (Tables 8-9).

TABLE 8
Notch and insulin co-operate to promote survival of mouse fetal central
nervous system stem cell cultures

| Treatment | Cell # |
|---|---|
| No Ins, no Dll4 | Lowest |
| Ins | Medium |
| Dll4 | High |
| Ins + Dll4 | Highest |

TABLE 9
Notch and insulin co-operate to promote survival of rat adult
subventricular zone central nervous system stem cell cultures

| Treatment | Cell # |
|---|---|
| No Ins, no Dll4 | Lowest |
| Ins | Medium |
| Dll4 | High |
| Ins+Dll4 | Highest |

In vivo, Notch and insulin also increase the number of dividing cells in the adult rat subventricular zone (SVZ) 5 days after a single injection It was determined that Notch and insulin co-operate to promote new (BrdU+) cells in vivo. Specifically, the reagents infused in the lateral ventricle of adult rats as a single injection. The rats were then given BrdU for 3 days and sacrificed on the 5^{th} day (Table 10).

TABLE 10
Notch and Insulin Increase the Number of Dividing cells

| Treatment | Cell # |
|---|---|
| No Ins, no Dll4 | Lowest |
| Ins | Medium |
| Dll4 | High |
| Ins+Dll4 | Highest |

It was determined that adult mouse subventricular zone central nervous system stem cell cultures from wild-type mice respond to D114 by generating twenty-four times more cells, whereas cultures from Hes3 knock-out mice respond to D114 by generating only three times more cells. Thus, in adult mouse neural stem cell cultures, D114 greatly promotes the survival of wild-type cells (23.7±1.7 fold increase), whereas it only slightly stimulates Hes3 knock-out culture survival (2.9±0.2 fold increase).

Pharmacological Notch activation is beneficial also to primary neural stem cell cultures from non-cancerous human brain. Primary human central nervous system neural stem cell were produced from pediatric subventricular zones. These cells expanded in FGF2, and upon FGF2 withdrawal differentiated to produce colonies containing neurons, astrocytes, and oligodendrocytes (see Table 11). D114 treatment generated significantly more of these colonies. FGF2 withdrawal and subsequent differentiation of central nervous system neural stem cell cultures (fetal mouse) regulated Hes3 expression and STAT3 expression and phosphorylation in neurons, astrocytes, oligodendrocytes.

TABLE 11
Expression of Hes3 after FGF2 withdrawal

| | Days of FGF2 withdrawal | | |
|---|---|---|---|
| | (Expression levels indicated) | | |
| Ab against: | 0 | 4-9 | 12 |

NEURONS

| | | | |
|---|---|---|---|
| Hes3 | High | Highest | Low |
| total STAT3 (K15) | High | High | Highest |
| C-term STAT3 (C20) | Medium | Very Low | Very High |
| STAT3-pS727 | Medium | Very Low | Very High |
| STAT3-pY705 | Non-detectable | Low | Medium |

ASTROCYTES

| | | | |
|---|---|---|---|
| Hes3 | High | Low | Low |

OLIGODENDROCYTES

| | | | |
|---|---|---|---|
| Hes3 | High | Highest | Low |

This result shows that Hes3 is a functional mediator of the pro-survival action of Notch stimulation.

Overall, this data demonstrates that Notch ligands and insulin work together to help activate the Notch receptor and also the insulin receptor. This means to help activate the insulin receptor (such as in diabetes), a possible way to facilitate this will be to treat the patient with Notch ligands. Conversely, to activate Notch receptors in diseases where Notch activation is faulty (such as for cerebral autosomal dominant arteriopathy (CADASIL)), insulin treatment might become a therapeutic strategy.

### Example 11

### Hes3 and AC133 Co-Localization in Human Glioblastoma

Human glioblastoma (GBM) is a brain tumor from which cancer stem cells can be identified by expression of AC133. AC133 is a transmembrane protein that exports many substances including chemotherapeutic agents to protect the cell expressing it; it is believed that when a tumor becomes non-responsive to chemotherapy, AC133 and related transporter proteins are responsible. Thus, chemotherapy often kills primary cancer cells but not cancer stem cells that express AC133.

Recent reports show the pattern of expression of AC133 in various cell types. Expression of AC133 is generally confined to cell processes and specialized membrane structures. A role for AC133 is determining whether a cell will divide symmetrically or asymmetrically has been proposed.

Human tumors were process for immunohistochemistry using anti-human Hes3 (Sigma); anti-human AC133 (Miltenyi biotechnology). In human tumors, such as glioblastomas, AC133 and Hes3 co-localize, strongly suggesting that Hes3 is also associated with these specialized membrane compartments. Thus, Hes3 expression can be used to diagnose glioblastoma, In addition, Hes3 can be involved in determining whether a cell will divide symmetrically or asymmetrically. Hes3 is regulated by the kinase LKB1, which is a regulator of cell polarity, a process important in symmetric/asymmetric division decisions.

Hes3 is also expressed in hemangioblastomas. Some of the cells that express Hes3 are megakaryocytes, the precursors of platelets. The megakaryocytes in hemangioblastomas are associated with poor prognosis.

Thus, a method is provided herein for diagnosing, or detecting the prognosis of, hemangioblastomas and glioblastomas. The methods including determining the expression of Hes3 and/or AC133 in a subject. In one embodiment, expression of Hes3 and/or AC133 indicates a poor prognosis for the subject. In another embodiment, determining the expression of Hes3 and/or AC133 indicates the presence of a hemangioblastoma or a glioblastoma. In some embodiments, the presence or absence of Hes3 and/or AC133 protein is assessed. In other embodiment, the presence or absence of nucleic acids encoding Hes3 and/or AC133 is assessed. Suitable methods are well known to those of skill in the art, and include the use of immunohistochemistry methods, Western blot techniques, reverse transcriptase polymerase chain reaction, Northern blot, and microarray analyses. One of skill in the art can readily perform assays to detect the presence and/or absence of Hes3 and/or AC133.

It will be apparent that the precise details of the methods or compositions described may be varied or modified without departing from the spirit of the described invention. We claim all such modifications and variations that fall within the scope and spirit of the claims below.

1-15. (canceled)

16. A method for increasing the proliferation of stem cells or precursor cells, comprising
contacting neuronal precursor cells with a therapeutically effective amount of (1) a Notch ligand and (2) a JAK inhibitor, a p38 inhibitor, or a combination thereof;
thereby increasing the proliferation of the neuronal precursor cells or stem cells.

17. The method of claim 16, wherein the Notch ligand is Delta.

18. The method of claim 16, wherein the Notch ligand is Jagged.

19. The method of claim 16, wherein the stem cells are mammalian embryonic stem cells.

20. The method of claim 19, wherein the mammalian embryonic stem cells are human embryonic stem cells.

21. The method of claim 16, wherein the precursor cells are somatic precursor cells.

22. The method of claim 21, wherein the somatic precursor cells are neuronal precursor cells or glial precursor cells.

23. The method of claim 21, wherein the somatic precursor cells are human.

24. The method of claim 16, further comprising contacting the cells with an effective amount of a growth factor.

25. The method of claim 24, wherein the growth factor is a fibroblast growth factor.

26. The method of claim 25, wherein the fibroblast growth factor is FGF-2.

27. The method of claim 16, further contacting the cells with an effective amount of insulin.

28. The method of claim 16, wherein the cells are pancreatic precursor cells.

29. A method for increasing the number of neuronal precursor cells or pancreatic precursor cells in a subject, comprising administering to the subject a therapeutically effective amount of a Notch ligand and a therapeutically effective amount of a growth factor, thereby increasing the number of neuronal precursor cells or pancreatic precursor cells in the subject.

30. The method of claim 29, wherein the Notch ligand is Delta.

31. The method of claim 29, wherein the growth factor is a fibroblast growth factor (FGF).

32. The method of claim 29, wherein the fibroblast growth factor is FGF-2, epidermal growth factor (EGF), insulin or platelet derived growth factor (PDGF).

33. The method of claim 29, wherein administering comprises local administration of a therapeutically effective amount of the Notch ligand and the growth factor.

34. The method of claim 33, wherein the method is a method for increasing the number of neuronal precursor cells, and wherein local administration comprises intraventricular injection.

35. The method of claim 29, comprising administering a therapeutically effective amount of Delta and a therapeutically effective amount of FGF-2.

36. The method of claim 29, wherein administering comprises delivering a single pulsatile dose of the growth factor and the Notch ligand.

37. The method of claim 29, wherein the method is a method for increasing the number of neuronal precursor cells, and wherein a stroke has occurred in the subject.

38. The method of claim 29, wherein the method is a method for increasing the number of neuronal precursor cells, and wherein the subject has a neurodegenerative disorder.

39. The method of claim 38, wherein the neurodegenerative disorder is Parkinson's disease or Alzheimer's disease.

40. The method of claim 28, wherein the growth factor is insulin.

41. The method of claim 40, wherein the Notch ligand is Delta.

42. The method of claim 40, wherein administering comprises local administration of a therapeutically effective amount of the Notch ligand and insulin.

43. The method of claim 40, wherein local administration comprises intraventricular injection.

44. The method of claim 40, wherein administering comprises delivering a single pulsatile dose of insulin and the Notch ligand.

45. The method of claim 40, wherein a stroke has occurred in the subject.

46. The method of claim 41, wherein the subject has a neurodegenerative disorder.

47. The method of claim 46, wherein the neurodegenerative disorder is Parkinson's disease or Alzheimer's disease.

48. A method for identifying an agent that increases the survival or proliferation of stem cells or somatic precursor cells, comprising
contacting a stem cell or a precursor cell with an effective amount of an agent of interest, wherein the stem cell or the somatic precursor cell expresses STAT3; and
determining the phosphorylation status of serine 727 of STAT3 in the stem cell or precursor cell,
wherein phosphorylation of serine 727 of STAT3 indicates that the agent increases the survival or proliferation of stem cells or somatic precursor cells.

49. The method of claim 48, further comprising
determining the phosphorylation status of tyrosine 705 of STAT 3 in the cell.

50. The method of claim 48, wherein the agent is a Notch agonist.

51. The method of claim 48, wherein the agent is a growth factor.

52. The method of claim 48, wherein the cell is a stem cell.

53. The method of claim 48, wherein the stem cell is an embryonic stem cell.

54. The method of claim 48, wherein the cell is a somatic precursor cell.

55. The method of claim 54, wherein the precursor cell is a neuronal precursor cell, a glial precursor cell, or a pancreatic precursor cell.

56. An isolated population of cells expressing nestin and STAT3, wherein serine 727 of STAT3 is phosphorylated.

57. The isolated population of cells of claim 56, wherein the cells express sonic hedgehog.

58. The isolated population of cells of claim 56, wherein the cells express Hes3.

59. The isolated population of cells of claim 56, wherein phosphorylation of tyrosine 705 of STAT 3 is not detectable.

60. The isolated population of cells of claim 56, wherein the cells express nucleostemin.

61. The isolated population of cells of claim 56, wherein the cells are STAT3Ser727⁺nestin⁺SHH⁺Nucl⁺ cells.

62. An isolated population of cells of claim 56, wherein the cells are STAT3 Ser727⁺STAT3Tyr705⁻nestin⁺SHH⁺Nucl⁺ cells.

63. A method for determining if a tumor is invasive, comprising
determining the phosphorylation status of serine 727 of STAT3 in a cell from the tumor,
wherein increased phosphorylation of serine 727 of STAT 3 in the cell as compared to a control indicates that tumor is invasive.

64. The method of claim 63, wherein the phosphorylation of serine 727 of STAT3 in the cell is compared to the phosphorylation of serine 727 in a non-transformed cell.

65. The method of claim 63, wherein the tumor is a tumor of the central nervous system.

66. The method of claim 63, further comprising assessing the presence of central nervous system stem cells.

67. A method for determining if an agent is of use for treating Alzheimer's disease, comprising
contacting a stem cell or a precursor cell with an effective amount of an agent of interest, wherein the stem cell or the precursor cell expresses STAT3; and
determining the phosphorylation status of serine 727 of STAT3 in the cell,
wherein phosphorylation of serine 727 of STAT3 indicates that the agent is of use in treating Alzheimer's disease.

68. The method of claim 67, wherein the agent comprises a Notch ligand.

69. The method of claim 67, wherein the agent further comprises a modulator of the p75 receptor
contacting a stem cell or a somatic precursor cell with an effective amount of an agent of interest, wherein the stem cell or the somatic precursor cell expresses STAT3; and
determining the phosphorylation status of serine 727 of STAT3 in the stem cell or somatic precursor cell,
wherein phosphorylation of serine 727 of STAT3 indicates that the agent increases the survival or proliferation of stem cells or somatic precursor cells.

70. The method of claim 29, wherein the cells are in vitro.

71. A method for detecting a hemangioblastoma or a glioblastoma in a subject, comprising
detecting the expression of Hes3 in a biological sample from the subject;
thereby detecting the presence of the hemangioblastoma or a glioblastoma in the subject.

72. The method of claim 71, wherein detecting the expression of Hes3 comprises detecting the presence of Hes3 protein.

73. The method of claim 72, wherein detecting the presence of Hes3 protein comprises the use of immunohistochemistry.

74. The method of claim 71, further comprising detecting the presence of AC133.
